(19) United States
(12) Patent Application Publication
(10) Pub. No.: US 2017/0360888 A1
(43) Pub. Date: Dec. 21, 2017

(54) METHODS FOR TREATING INFLAMMATORY ARTHRITIS

(71) Applicant: NEW YORK UNIVERSITY, New York, NY (US)

(72) Inventors: Bruce CRONSTEIN, New York, NY (US); Edward A. FISHER, Scarsdale, NY (US); Kathryn J. MOORE, Westfield, NJ (US); Aranzazu MEDIERO MUNOZ, New York, NY (US)

(21) Appl. No.: 15/536,195

(22) PCT Filed: Dec. 16, 2015
(86) PCT No.: PCT/US15/65961

§ 371 Date: Jun. 15, 2017

Related U.S. Application Data
(60) Provisional application No. 62/092,971, filed on Dec. 17, 2014.

(51) Int. Cl. A61K 38/18; C07K 16/28; C07K 16/22; C12N 15/113; A61K 39/00

(52) U.S. Cl. CPC A61K 38/18; C07K 16/2803; C07K 16/22; A61K 2039/505; C12N 15/1136; C12N 2310/14

(57) ABSTRACT

The invention provides methods and compositions for inhibiting, reducing or slowing inflammation or inflammatory arthritis, for treating inflammatory arthritis such as rheumatoid arthritis, psoriatic arthritis, and a spondyloarthropathy, for inhibiting, reducing or slowing osteoclast differentiation, function, or biological activity, for inhibiting or reducing persistence or accumulation of macrophages at an inflamed site or in an inflamed tissue or for promoting or increasing the efflux of macrophages from an inflamed site or an inflamed tissue, for increasing, stimulating, or promoting lymphocyte cell adhesion, for increasing, stimulating, or promoting lymphocyte efflux from a site of inflammation or from an inflamed tissue, and for decreasing, inhibiting, or reducing a lymphocyte cellular response by inhibiting, inhibiting the biological activity of or antagonizing an axonal guidance protein or a receptor thereof. The axonal guidance protein may be a netrin such as netrin-1, and its receptor may be unc5b.

### FIELD OF THE INVENTION

The present invention relates to methods and compositions for treating and inhibiting inflammation and inflammatory arthritis.

### BACKGROUND OF THE INVENTION

### Axon Guidance Molecules

A combination of genetic and biochemical methods has led to the discovery of several important classes of axon guidance molecules and their receptors. Netrins are secreted molecules that can act to attract or repel axons by binding to their receptors, DCC and UNC5. Slits also known as Sli are secreted proteins that normally repel growth cones by engaging Robo (Roundabout) class receptors. Ephrins are cell surface molecules that activate Eph receptors on the surface of other cells. This interaction can be attractive or repulsive. In some cases, Ephrins can also act as receptors by transducing a signal into the expressing cell, while Ephs act as the ligands. Signaling into both the Ephrin- and Eph-bearing cells is called “bi-directional signaling.” Semaphorins occur as many types and are primarily axonal repellents, and activate complexes of cell-surface receptors called Plexins and Neuropilins. Cell adhesion molecules (CAMs) are integral membrane proteins mediating adhesion between growing axons and eliciting intracellular signalling within the growth cone. CAMs are the major class of proteins mediating correct axonal navigation of axons growing on axons (fasciculation). There are two CAM subgroups: IgSF-CAMs (belonging to the immunoglobulin superfamily) and Cadherins (Ca-dependent CAMs).

In addition, many other classes of extracellular molecules are used by growth cones to navigate properly including developmental morphogens, such as BMPs, Wnts, Hedgehog, and FGFs, extracellular matrix and adhesion molecules such as laminin, tenascins, proteoglycans, N-CAM, and L1, growth factors like NGF, and neurotransmitters and modulators like GABA.

Growing axons rely on a variety of guidance cues in deciding upon a growth pathway. The growth cones of extending axons process these cues in an intricate system of signal interpretation and integration, in order to insure appropriate guidance. Adhesive cues provide physical interaction with the substrate necessary for axon protrusion. These cues can be expressed on glial and neuronal cells the growing axon contacts or be part of the extracellular matrix. Examples are laminin or fibronectin, in the extracellular matrix, and cadherins or Ig-family cell-adhesion molecules, found on cell surfaces. Tropic cues act as attractants or repellents and cause changes in growth cone motility by acting on the cytoskeleton through intracellular signaling. For example, Netrin plays a role in guiding axons through the midline, acting as both an attractant and a repellent. While Semaphorin3A, helps axons grow from the olfactory epithelium to map different locations in the olfactory bulb. Modulatory cues influence the sensitivity of growth cones to certain guidance cues. For instance, neurotrophins can make axons less sensitive to the repellent action of Semaphorin3A.

Given the abundance of these different guidance cues it was previously believed that growth cones integrate various information by simply summing the gradient of cues, in different valences, at a given point in time, to making a decision on the direction of growth. However, studies in vertebrate nervous systems of ventral midline crossing axons, has shown that modulatory cues play a crucial part in tuning axon responses to other cues, suggesting that the process of axon guidance is nonlinear. For examples, commisurial axons are attracted by netrin and repelled by slit. However, as axons approach the midline, the repellent action of Slit is suppressed by Robo-3/Rig-1 receptor. Once the axons cross the midline, activation of Robo by Slit silences Netrin-mediated attraction, and the axons are repelled by Slit.

### Netrins

The netrin family is composed mostly of secreted proteins which serve as bifunctional signals: attracting some neurons while repelling others during the development of brain. Expressed in the midline of all animals possessing bilateral symmetry, they can act as long or short range signals during neurogenesis. In order to carry out their functions, netrins interact with specific receptors: DCC or UNC-5 depending on whether they are trying to attract or repel neurons respectively. There is a high degree of conservation in the secondary structure of netrins, which has several domains which are homologous with laminin at the amino terminal end. The C-terminal domain is where most of the variation is found between species and contains different amino acids which allow interaction with specific proteins in extracellular matrix or on cell surface. The differences in terms of structure and function have led to the identifications of several different types of netrins including netrin-1, netrin-3, and netrins-G.

Netrin-1 (Ntn1) is found in the floor plate and neuroepithelial cells of the ventral region of the spinal cord, as well as other locations in the nervous system including the somatic mesoderm, pancreas and cardiac muscle. Its main role is in axonal guidance, neuronal migration and morphogenesis of different branching structures. Mice with mutations in the netrin-1 gene were observed to be lacking in forebrain and spinal cord commissural axons. Netrin-3 is different from other netrins. While expressed during development of the peripheral nervous system in the motor, sensory and sympathetic neurons, it is very limited in the central nervous system. Studies with netrin-3 have noticed a reduced ability to bind with DCC receptors when compared with netrin-1. This suggests that it mainly operates through other receptors. Netrins-G are secreted but remain bound to the extracellular surface of the cell membrane through Glycophosphatidylinositol (GPI). They are expressed predominantly in the central nervous system in places such as the thalamus and mitral cells of the olfactory bulb. (Park, et al., J Clin Invest, 2009, 119: 136-145) They do not bind to DCC or UNC-5 and instead bind to ligand NGL-1, which results in an intracellular transduction cascade. The two versions, netrins-G1 and netrins-G2, are found only in vertebrates. It is believed that they evolved independently of other netrins in order to facilitate the construction of the brain.

DCC and UNC-5 proteins carefully mediate netrin-1 responses. The UNC-5 protein is mainly involved in signaling repulsion. DCC, which is implicated in attraction, can also serve as a co-factor in repulsion signaling when far away from the source of netrin-1. DCC is highly expressed in the central nervous system and associated with the basal lamina of epithelia cells. In the absence of netrin-1, these receptors are known to induce apoptosis.

### Inflammation

Inflammation is a process regulated by the coordinated actions of soluble mediators (including chemokines, cytokines, small molecules (e.g. prostaglandins) and other protein mediators (e.g. immunoglobulins, complement proteins)), interacting with cellular receptors and targets to promote the recruitment of inflammatory cells to a specific site where they mediate tissue injury, repair and host defense. Recent studies indicate that additional signals can prevent cells from exiting an inflamed site to perpetuate tissue injury. Netrin-1 has been identified as such a molecule (van Gils et al., Nature Immunology 2012; 13(2):136-43, van Gils et al., Arteriosclerosis, Thrombosis, and Vascular Biology 2013; 33(5):911-9, Ramkhelawon et al., Nature Medicine 2014; 20(4):377-84).

Ntn1 has multiple roles in inflammation. Ntn1 has been shown to be both a positive and negative regulator of cell migration and inflammation. Recent studies have identified roles for Ntn1 outside the nervous system, in angiogenesis and inflammation. This secreted laminin-related molecule was shown to function as a leukocyte-guidance molecule that inhibits the migration of monocytes, neutrophils and lymphocytes by activation of its receptor Unc5b (Ly et al., Proceedings of the National Academy of Sciences of the United States of America 2005; 102(41):14729-34, Mirakaj et al., American Journal of Respiratory and Critical Care Medicine 2010; 181(8):815-24, Rosenberger et al., Nature Immunology 2009; 10(2):195-202).

Although initial studies suggested that Ntn1 suppressed inflammation by reducing leukocyte entry into tissues, more recent studies indicate that Ntn1 expression by macrophages promotes inflammation. Ntn1 expression by epithelial/endothelial cells, or Ntn1 infusion, protects the kidney against hypoxia and other forms of acute injury, diminishes pulmonary inflammation, and inhibits pancreatitis, inflammatory bowel disease and ischemic injury to the gut, corneal inflammation and peritonitis (Chen et al., PloS one 2012; 7(9):e46201; Grenz et al., PloS one 2011; 6(5):e14812; Han et al., Investigative Ophthalmology & Visual Science 2012; 53(3):1285-95; Ly et al., Proceedings of the National Academy of Sciences of the United States of America 2005; 102(41):14729-34; Mirakaj et al, Journal of Immunology 2011; 186(1):549-55; Mirakaj et al., American Journal of Respiratory and Critical Care Medicine 2010; 181(8):815-24; Tadagavadi et al., Journal of Immunology 2010;185(6):3750-8; Wang et al., American Journal of Physiology Renal Physiology 2008; 294(4):F739-47; Rosenberger et al., Nature Immunology 2009; 10(2):195-202; 23; Carney et al., Nature Reviews Nephrology 2012. doi: 10.1038/nrneph.2012.228. PubMed PMID: 23090448; Grenz et al., Current Opinion in Critical Care 2012; 18(2):178-85; Mohamed et al., The American Journal of Pathology 2012; Moon et al., Journal of Neuroimmunology 2006; 172(1-2):66-72; Mutz et al., Critical Care 2010; 14(5):R189; Paradisi et al., Proceedings of the National Academy of Sciences of the United States of America 2009; 106(40):17146-51; Rajasundari et al., Laboratory Investigation; A Journal of Technical Methods and Pathology 2011; 91(12):1717-26). In many of these inflammatory models the anti-inflammatory effects of Ntn1 have been ascribed to the anti-inflammatory effects of adenosine A_{2B} receptors (Aherne et al., Gut 2012; 61(5):695-705, Mirakaj et al, Journal of Immunology 2011; 186(1):549-55; Rosenberger et al., Nature Immunology 2009; 10(2):195-202, 24). Interestingly, adenosine A_{2B} receptor stimulation was recently reported to reverse the anti-inflammatory effects of methotrexate treatment in the adjuvant arthritis model (Teramachi et al., Laboratory Investigation; A Journal of Technical Methods and Pathology 2011; 91(5):719-31), a finding which suggests that the anti-inflammatory effects of adenosine A_{2B} receptors and, by extension, Ntn1 may differ dramatically depending on the site of inflammation. In contrast to the anti-inflammatory effects of Ntn1 in models of gut, eye and peritoneal inflammation, Ntn1 was recently identified as an endogenous promoter of atherosclerosis and obesity-induced insulin resistance by autocrine inhibition of macrophage emigration from atherosclerotic plaques and visceral adipose tissue, respectively (van Gils et al., Nature immunology 2012; 13(2):136-43), 12). Fibroblast-like synoviocytes (FLS) from patients with rheumatoid arthritis express increased Ntn1 and unc5b levels and Ntn1 inhibits migration of FLS (Schubert et al., International Journal of Immunopathology and Pharmacology 2009; 22(3):715-22), a finding interpreted as pro-inflammatory.

### Inflammatory Arthritis and Joint Destruction

Joint destruction, the final result of inflammatory arthritis, results in pain and increasing disability. Synovial fibroblasts, cells of the innate and adaptive immune system, and osteoclasts, the myeloid-derived cells that mediate bone destruction in inflammatory arthritis, all participate in destruction of the tissues of the joint. The destructive process is further orchestrated by inflammatory cytokines and chemokines, antibodies and small molecule mediators secreted by the inflammatory cells present at the site of injury. The recruitment of these cells to the joint requires the coordinated expression of cytokines, adhesion molecules and chemoattractants at the inflamed site. Recent studies indicate that chemotropic proteins, such as netrin-1 (Ntn1), increase inflammation by preventing inflammatory cell egress from inflamed sites (van Gils et al., Nature immunology 2012; 13(2):136-43).

### SUMMARY OF THE INVENTION

The invention relates to the application and use of modulators of axonal guidance, including antagonists or inhibitors of axonal guidance proteins or their biological receptors, to inhibit inflammation and treat inflammatory arthritis.

In a first aspect, the invention provides a method for inhibiting, reducing or slowing inflammation or inflammatory arthritis by inhibiting, inhibiting the biological activity of or antagonizing an axonal guidance protein or a receptor thereof. The method may feature administering to a subject a therapeutically effective amount of an agent effective to inhibit or reduce the biological activity of an axonal guidance protein or a receptor of the axonal guidance protein, or an analog, derivative or combination thereof

In a second aspect, the invention provides a method for treating a disease caused all or in part by or characterized by inflammation such as, for instance, inflammatory arthritis. The method may feature inhibiting inflammation or increasing or promoting bone density by inhibiting, inhibiting the biological activity of or antagonizing an axonal guidance protein or a receptor thereof. The method may feature administering to a subject a therapeutically effective amount of an agent effective to inhibit or reduce the biological activity of an axonal guidance protein or a receptor of the axonal guidance protein, or an analog, derivative or combination thereof. The inflammatory arthritis may be, for instance, rheumatoid arthritis, psoriatic arthritis, or a spondyloarthropathy.

In a third aspect, the invention provides a method for inhibiting, reducing or slowing osteoclast differentiation, function, or biological activity by inhibiting, inhibiting the biological activity of or antagonizing an axonal guidance protein or a receptor thereof. The method may feature administering to a subject a therapeutically effective amount of an agent effective to inhibit or reduce the biological activity of an axonal guidance protein or a receptor of the axonal guidance protein, or an analog, derivative or combination thereof

In a fourth aspect, the invention provides a method for inhibiting or reducing persistence or accumulation of macrophages at an inflamed site or in an inflamed tissue or for promoting or increasing the efflux of macrophages from an inflamed site or inflamed tissue by inhibiting, inhibiting the biological activity of or antagonizing an axonal guidance protein or a receptor thereof. The method may feature administering to a subject a therapeutically effective amount of an agent effective to inhibit or reduce the biological activity of an axonal guidance protein or a receptor of the axonal guidance protein, or an analog, derivative or combination thereof

In a fifth aspect, the invention provides a method for increasing, stimulating, or promoting lymphocyte cell adhesion by inhibiting, inhibiting the biological activity of or antagonizing an axonal guidance protein or a receptor thereof. The method may feature administering to a subject a therapeutically effective amount of an agent effective to inhibit or reduce the biological activity of an axonal guidance protein or a receptor of the axonal guidance protein, or an analog, derivative or combination thereof. The lymphocyte may be, for instance, a T cell, including a T helper cell, such as a Th1, Th2, Th17, or Treg cell.

In a sixth aspect, the invention provides a method for increasing, stimulating, or promoting lymphocyte efflux from a site of inflammation or from an inflamed tissue by inhibiting, inhibiting the biological activity of or antagonizing an axonal guidance protein or a receptor thereof. The method may feature administering to a subject a therapeutically effective amount of an agent effective to inhibit or reduce the biological activity of an axonal guidance protein or a receptor of the axonal guidance protein, or an analog, derivative or combination thereof. The lymphocyte may be, for instance, a T cell, including a T helper cell, such as a Th1, Th2, Th17, or Treg cell.

In a seventh aspect, the invention provides a method for decreasing, inhibiting, or reducing a lymphocyte cellular response by inhibiting, inhibiting the biological activity of or antagonizing an axonal guidance protein or a receptor thereof. The method may feature administering to a subject a therapeutically effective amount of an agent effective to inhibit or reduce the biological activity of an axonal guidance protein or a receptor of the axonal guidance protein, or an analog, derivative or combination thereof. The lymphocyte may be, for instance, a T cell, including a T helper cell, such as a Th1, Th2, Th17, or a Treg cell.

For each of these above aspects, the axonal guidance protein may be, for instance, one of sema3E, plexin, and a netrin such as netrin-1. Also, for each of these above aspects the agent effective to inhibit or reduce the biological activity of an axonal guidance protein or a receptor of the axonal guidance protein, such as for instance netrin-1 or its receptor unc5b or DCC or semaphorin3E or its receptor plexinD1, may be, for instance, an antibody such as a monoclonal antibody, a peptide, a nucleic acid, for instance, an interfering RNA such as siRNA, or a small molecule.

In some instances the agent effective to inhibit or reduce the biological activity of an axonal guidance protein or a receptor of the axonal guidance protein, or an analog, derivative or combination thereof, may be administered in combination with one or more drugs useful in inhibiting inflammation. Such other compounds may be, for instance, anti-inflammatory compounds, bisphosphonates or growth factors. The subject may be a mammal including a human, and the subject may be suffering from or experiencing inflammation such as an inflammatory arthritis. As well, the subject may be in need of treatment for the inflammation or inflammatory arthritis.

In an eighth aspect, the present invention provides a pharmaceutical composition comprising an agent effective to inhibit or reduce the biological activity of an axonal guidance protein or a receptor of the axonal guidance protein, or an analog, derivative or combination thereof alone or in combination with one or more second compound or agent effective for inhibiting inflammation or treating an inflammatory arthritis such as an inflammatory arthritis including, for instance, rheumatoid arthritis. The axonal guidance protein may be, for instance, one of sema3E, plexin, and a netrin such as netrin-1. The agent effective to inhibit or reduce the biological activity of an axonal guidance protein or a receptor of the axonal guidance protein, such as for instance netrin-1 or its receptor unc5b or DCC or semaphorin3E or its receptor plexinD1, may be, for instance, an antibody such as a monoclonal antibody, a peptide, a nucleic acid, for instance, an interfering RNA such as siRNA, or a small molecule.

The agent effective to inhibit or reduce the biological activity of an axonal guidance protein or a receptor of the axonal guidance protein and the one or more second compound or agent may be formulated and administered alone or together. The pharmaceutical composition(s) comprising the agent effective to inhibit or reduce the biological activity of an axonal guidance protein or a receptor of the axonal guidance protein and the one or more second compound or agent may be administered concurrently or sequentially.

The pharmaceutical compositions may be delivered topically, orally or parenterally. They may be delivered via an intravenous route, an intramuscular route, or a subcutaneous route. They may be delivered as an immediate release formulation or as a slow or sustained release formulation. Likewise, they may be delivered as suppositories, enemas or via aerosol.

In some embodiments, the pharmaceutical composition comprising an agent effective to inhibit or reduce the biological activity of an axonal guidance protein or a receptor of the axonal guidance protein may also contain one or more drugs such as anti-inflammatory agents. Also, the agent effective to inhibit or reduce the biological activity of an axonal guidance protein or a receptor of the axonal guidance protein, such as, for instance, netrin-1 or its receptor unc5b may be, for instance, an antibody such as a monoclonal antibody, a peptide, a nucleic acid, for instance, an interfering RNA such as siRNA, or a small molecule.

Other objects and advantages will become apparent to those skilled in the art from a review of the following description which proceeds with reference to the following illustrative drawings.

### BRIEF DESCRIPTION OF THE FIGURES

FIG. 1 demonstrates that antibodies to Ntn1 and unc5b, but not DCC, diminish arthritis severity in the KBxN serum-induced arthritis model by reducing paw thickness. Mice were injected IP with 350 microliters of serum from KBxN arthritic mice and anti-ntn1, -unc5b or -DCC antibodies (100 ml) or saline. Severity was assessed at day 0, 10, 16 and 21, and each point represents the mean (±SD) of severity of 10 (Control and anti-ntn1 antibody treated) or 5 mice. The maximum severity score is 16 for each mouse. ***p<0.001, ANOVA with repeated measures.

FIG. 2 demonstrates that antibodies to Ntn1 and unc5b, but not DCC, diminish arthritis severity in the KBxN serum-induced arthritis model by reducing or slowing inflammation. Mice were injected IP with 350 microliters of serum from KBxN arthritic mice and anti-ntn1, -unc5b or -DCC antibodies (100 ml) or saline. Severity was assessed and each point represents the mean (+SD) of severity of 10 (Control and anti-ntn1 antibody treated) or 5 mice. The maximum severity score is 16 for each mouse. Inflammation is scored 0-4 on 4 paws. ***p<0.001, ANOVA with repeated measures. A) represents pay thickness in millimeters, and B) represents inflammation.

FIG. 3 represents histological differences of ankle tissue specimens between (A) a control and (B) a mouse 2 weeks post injection of 350 microliters of serum from KBxN arthritic mice and anti-ntn1.

FIG. 4 further demonstrates that antibodies to Ntn1 and unc5b, but not DCC diminish arthritis severity in the KBxN serum-induced arthritis model. Mice were injected IP with 350 μl of serum from KBxN arthritic mice and anti-Ntn1, -unc5b or -DCC antibodies (100 μl) or saline. Severity was assessed and each point represents the mean (±SD) of severity of 10 (Control and anti-Ntn1 antibody treated) or 5 mice. ***p<0.001, ANOVA with repeated measures.

FIG. 5 demonstrates that Ntn1 is highly expressed in inflammatory infiltrates in wear particle-induced osteolysis. A) Ntn1, Unc5b and DCC immunostaining in a osteolysis mouse model. Bone osteolysis was induced by UHMWPE particles. Number of positive cells was calculated as mean ±SEM for 5 slides per condition (n=3 each). B) Ntn1 immunostaining in human tissue biopsies from implant revision and primary implants. ***p<0.001, compared to untreated, ANOVA. All images are the same magnification.

FIG. 6 demonstrates that treatment with Ntn1 or UncSb receptor antibodies diminish inflammatory infiltrates in wear particle-induced osteolysis. Calvaria were processed and microCT and immunohistologic staining carried out on calvaria from UHMWPE-exposed mice, in the presence or absence of Netrin-1, UncSb receptor and DCC receptor antibodies A) Representative high resolution microCT images. 3D images reconstruction of the calvaria revealed decreased bone loss and pitting in Netrin-1 or Unc5b receptor antibodies treated animals. Digital morphometric analysis of microCT images was performed. All data are expressed as means±SEM (n=5).

***p<0.001, ANOVA.

FIG. 7 demonstrates in vitro characterization of Ntn1^{-/-} bone marrow derived osteoclast cultures. A) WT and Ntn^{+/-} mice osteoclast primary culture cells were fixed and stained for TRAP after being cultured with Netrin-1 alone or combined with Unc5b or DCC antibodies. TRAP-positive cells containing three or more nuclei were counted as osteoclasts. B) Toluidine Blue staining to assess osteoclast activity. Non-adherent cells were treated with Ntn1 alone or combined with Unc5b or DCC antibodies. C) Dose response effect of Netrin-1 blockade on osteoclast differentiation. Netrin-1, Unc5b and DCC antibodies were exposed to cultures at various time points after the start of the cultures. WT mouse osteoclast primary culture cells stained with TRAP to counteract osteoclast. All results are expressed as means (±SEM) of 6 independent cultures.***p<0.001, *p<0.5 related to WT (ANOVA).

FIG. 8 demonstrates that morphometric examination of long bones in 5 month old WT and Ntn1^{-/-} mice. A) Whole-body dual x-ray absorptiometry (DXA) scanning to assessed the bone mineral density (BMD) (gm/cm²) of the whole skeletons of Ntn1^{-/-} and wild-type (WT) mice (n=9 each). B) Representative high resolution microCT images. 3D images reconstruction of the femurs revealed increased bone mass in Ntn1^{-/-} mice compared with their WT littermates (n=5each). C) Digital morphometric analysis of microCT images from WT and Ntn1^{+/-} mice. All data are expressed as means (±SEM). ***p<0.001, *p<0.05 vs WT (Student's t test or ANOVA).

FIG. 9 depicts MØ Ntn1 DNA in Ntn1^{fl/fl}UBCcre− or cre+ mice treated with Tamoxifen (TX).

FIG. 10 demonstrates that Ntn1 promotes MØ polarization. A) Expression of M1 and M2 markers in BMDM (M0) treated with 1 μg/ml LPS+100 ng/ml IFNγ to polarize to M1 or with 10 ng/ml IL-4 to polarize to M2 in the presence/absence of 250 ng/ml Ntn1 for 24 h. B) Quantification of NFkB activation in THP1-Blue reporter cells treated with vehicle, 1 ng/ml LPS, 10 ng/ml s100b or 250 uM palmitate in the presence/absence of Ntn1 (250 ng/ml; 24 h). Data are the mean±sem and representative of 2 experiments. *P<0.05

FIG. 11 demonstrates that Unc5b and Netrin-1 inhibit T cell adhesion. A) Primary human T cells were isolated from peripheral blood and the surface levels of the activation marker CD69 were recoded by FACS while the expression levels of Unc5b were assessed by western blotting. n=8. B) Primary human T cells were subjected to adhesion assay before and after stimulation with anti-CD antibodies, and with or without pretreatment with recombinant Netrin-1. n=3.

FIG. 12 represents Seahorse XF analysis of M1/M2 MØ.

FIG. 13 demonstrates that Ntn1 and Unc5b are increased during osteoclast differentiation. Ntn1 and Unc5b interactions lead to RhoA phosphorylation and FAK activation. A) Fold change in Ntn1, Unc5b and DCC mRNA in murine M-CSF/RANKL osteoclast precursors during the seven days of osteoclast differentiaion. B) Ntn1 expression and secretion and Unc5b and DCC expression were analyzed 24 hours after RANKL stimulation in murine BMCs cells. C) Murine BMCs were treated with 30 ng/ml RANKL together with recombinant Ntn1 alone or in combination with Unc5b or DCC antibodies. RhoA phosphorylation was analyzed 15 minutes after stimulation by western blot of lysates. D) FAK expression was analyzed 15 minutes after stimulation by western blot of lysates. To normalize for protein loading, the membranes were re-probed with RhoA or actin respectively and results normalized appropriately. E) Cell extracts were immunoprecipitated with anti-Unc5b antibody. The immunoprecipitates were then analyzed by immunoblotting with anti-Neogenin, anti-LARG and anti-RGMa antibodies. The figure shows representative data from one of four experiments. F) Proposed intracellular pathway activated by Ntn1/Unc5b to promote changes in RhoA cytoskeleton. The results were expressed as the means of four independent experiments. ***p<0.001, **p<0.01, *p<0.5 vs. non-stimulated control.

FIG. 14 demonstrates that stimulation of Unc5b promotes cells fusion associated with DC-STAMP. A) Murine BMCs were treated with 30 ng/ml RANKL together with recombinant Netrin-1. Cell extracts were immunoprecipitated with anti-Unc5b antibody. The immunoprecipitates were then analyzed by immunoblotting with anti-DC-STAMP antibody. The figure shows representative data from one of four experiments. B) RAW264.7 cells were stably transduced with scrambled or Netrin-1, Unc5b or DC-STAMP shRNA and treated with 50 ng/ml RANKL for four days. DC-STAMP immunostaining is shown in red and Unc5b immunolocalization in shown red. Images were taken at an original

### DETAILED DESCRIPTION OF THE INVENTION

Netrin-1 is an axonal guidance protein which acts as a chemorepulsant. Prior studies have indicated that netrin-1 is produced by activated macrophages and may play a role in the pathogenesis of atherosclerotic plaque, among other inflammatory lesions.

The present invention is based in part upon that discovery that it is possible to inhibit inflammation and treat inflammatory arthritis including rheumatoid arthritis using agents that either neutralize or block netrin-1 or agents which block its receptors such as unc5b. This approach to therapy is useful for the treatment of inflammatory arthritis such as rheumatoid arthritis, etc.

Before the present methods and treatment methodology are described, it is to be understood that this invention is not limited to particular methods and experimental conditions described, as such methods and conditions may vary. It is also to be understood that the terminology used herein is for purposes of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth in their entirety.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entireties.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### Definitions

The terms used herein have the meanings recognized and known to those of skill in the art, however, for convenience and completeness, particular terms and their meanings are set forth below.

“Agent” refers to all materials that may be used to prepare pharmaceutical and diagnostic compositions, or that may be compounds such as small synthetic or naturally derived organic compounds, nucleic acids, polypeptides, antibodies, fragments, isoforms, variants, or other materials that may be used independently for such purposes, all in accordance with the present invention.

By “agonist” is meant a substance that binds to a specific receptor and triggers a response in a cell. It mimics the action of an endogenous ligand (such as hormone or neurotransmitter) that binds to the same receptor. A “full agonist” binds (has affinity for) and activates a receptor, displaying full efficacy at that receptor. One example of a drug that acts as a full agonist is isoproterenol which mimics the action of acetylcholine at β adrenoreceptors. A “partial agonist” (such as buspirone, aripiprazole, buprenorphine, or norclozapine) also binds and activates a given receptor, but has only partial efficacy at the receptor relative to a full agonist. A “partial agonist” may also be considered a ligand that displays both agonistic and antagonistic effects—when both a full agonist and partial agonist are present, the partial agonist actually acts as a competitive antagonist, competing with the full agonist for receptor occupancy and producing a net decrease in the receptor activation observed with the full agonist alone. A “co-agonist” works with other co-agonists to produce the desired effect together. An antagonist blocks a receptor from activation by agonists. Receptors can be activated or inactivated either by endogenous (such as hormones and neurotransmitters) or exogenous (such as drugs) agonists and antagonists, resulting in stimulating or inhibiting a biological response. A ligand can concurrently behave as agonist and antagonist at the same receptor, depending on effector pathways.

The potency of an agonist is usually defined by its EC₅₀ value. This can be calculated for a given agonist by determining the concentration of agonist needed to elicit half of the maximum biological response of the agonist. Elucidating an EC₅₀ value is useful for comparing the potency of drugs with similar efficacies producing physiologically similar effects. The lower the EC₅₀, the greater the potency of the agonist and the lower the concentration of drug that is required to elicit a maximum biological response.

“Antagonist” refers to an agent that down-regulates (e.g., suppresses or inhibits) at least one bioactivity of a protein. An “antagonist” or an agent that “antagonizes” may be a compound which inhibits or decreases the interaction between a protein and another molecule, e.g., a target peptide or enzyme substrate. An antagonist may also be a compound that down-regulates expression of a gene or which reduces the amount of expressed protein present. Methods for assessing the ability of an agent to “antagonize” or “inhibit” an axonal guidance protein or a receptor thereof are known to those skilled in the art.

“Analog” as used herein, refers to a chemical compound, a nucleotide, a protein, or a polypeptide that possesses similar or identical activity or function(s) as the chemical compounds, nucleotides, proteins or polypeptides having the desired activity and therapeutic effect of the present invention (e.g. to treat or prevent bone disease, or to modulate osteoclast differentiation), but need not necessarily comprise a compound that is similar or identical to those compounds of the preferred embodiment, or possess a structure that is similar or identical to the agents of the present invention.

“Derivative” refers to the chemical modification of molecules, either synthetic organic molecules or proteins, nucleic acids, or any class of small molecules such as fatty acids, or other small molecules that are prepared either synthetically or isolated from a natural source, such as a plant, that retain at least one function of the active parent molecule, but may be structurally different. Chemical modifications may include, for example, replacement of hydrogen by an alkyl, acyl, or amino group. It may also refer to chemically similar compounds which have been chemically altered to increase bioavailability, absorption, or to decrease toxicity. A derivative polypeptide is one modified by glycosylation, pegylation, or any similar process that retains at least one biological or immunological function of the polypeptide from which it was derived.

A “small molecule” refers to a molecule that has a molecular weight of less than 3 kilodaltons (kDa), preferably less than about 1.5 kilodaltons, more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is normally an organic compound (or organic compound complexed with an inorganic compound (e.g., metal)) that has a molecular weight of less than 3 kilodaltons, and preferably less than 1.5 kilodaltons, and more preferably less than about 1 kDa.

“Diagnosis” or “screening” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

The concept of “combination therapy” is well exploited in current medical practice. Treatment of a pathology by combining two or more agents that target the same pathogen or biochemical pathway sometimes results in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect can be synergistic (the efficacy of the combination is greater than the sum of the effects of each drug given alone). As used herein, the term “combination therapy” means the two compounds can be delivered in a simultaneous manner, e.g. concurrently, or one of the compounds may be administered first, followed by the second agent, e.g. sequentially. The desired result can be either a subjective relief of one or more symptoms or an objectively identifiable improvement in the recipient of the dosage.

“Modulation” or “modulates” or “modulating” refers to up regulation (i.e., activation or stimulation), down regulation (i.e., inhibition or suppression) of a response, or the two in combination or apart. As used herein, an inflammation “modulator” or “modulating” compound or agent is a compound or agent that modulates at least one biological marker or biological activity characteristic of inflammation.

As used herein, the term “candidate compound” or “test compound” or “agent” or “test agent” refers to any compound or molecule that is to be tested. As used herein, the terms, which are used interchangeably, refer to biological or chemical compounds such as simple or complex organic or inorganic molecules, peptides, proteins, oligonucleotides, polynucleotides, carbohydrates, or lipoproteins. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed. As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action.

“Treatment” or “treating” refers to therapy, prevention and prophylaxis and particularly refers to administering medicine or performing medical procedures on a patient, for either prophylaxis (prevention) or to cure or reduce the extent of or likelihood of occurrence of the infirmity or malady or condition or event. In the present invention, the treatments using the agents described may be provided to slow or halt symptoms, pathology or the disease process.

“Subject” or “patient” refers to a mammal, preferably a human, in need of treatment for a condition, disorder or disease.

An “amount sufficient to inhibit inflammation” refers to the amount of the agent sufficient to block or inhibit the activity of cells involved in or biological markers indicative of an inflammatory response.

In a specific embodiment, the term “about” means within 20%, preferably within 10%, and more preferably within 5% or even within 1%.

An “effective amount” or a “therapeutically effective amount” is an amount sufficient to decrease, ameliorate or prevent the symptoms associated with the conditions disclosed herein, including inflammation, edema, fever, osteolysis, etc. For example, an “effective amount” for therapeutic uses is the amount of the composition comprising an active compound herein required to provide reversal or inhibition of inflammation, osteolysis, etc. Such effective amounts may be determined using routine optimization techniques and are dependent on the particular condition to be treated, the condition of the subject, the route of administration, the formulation, and the judgment of the practitioner and other factors evident to those skilled in the art. The dosage required for the compounds of the invention is that which reduces an inflammatory response or chronic inflammation. The “effective amount” or “therapeutically effective amount” may range from about 1 mg/Kg to about 200 mg/Kg in vivo, or more preferentially from about 10 mg/Kg to about 100 mg/Kg, and most preferentially from about 25 mg/Kg to about 50 mg/Kg in vivo.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

Binding compounds can also be characterized by their effect on the activity of the target molecule. Thus, a “low activity” compound has an inhibitory concentration (IC₅₀) (for inhibitors or antagonists) or effective concentration (EC₅₀) (applicable to agonists) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured.

An individual “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of inflammation including chronic inflammation and/or bone or joint damage or destruction.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Differentiate” or “differentiation” as used herein, generally refers to the process by which precursor or progenitor cells differentiate into specific cell types. In the present invention, the term refers to the process by which pre-osteoclasts become osteoclasts. Differentiated cells can be identified by their patterns of gene expression and cell surface protein expression. As used herein, the term “differentiate” refers to having a different character or function from the original type of tissues or cells. Thus, “differentiation” is the process or act of differentiating. The term “Osteoclast Differentiation” refers to the process whereby osteoclast precursors in the bone marrow become functional osteoclasts.

“Osteoclastogenesis” refers to osteoclast generation, which is a multi-step process that can be reproduced in vitro. Earlier in vitro osteoclastogenesis systems used mixtures of stromal or osteoblastic cells together with osteoclast precursors from bone marrow (Suda, et al., Methods Enzymol., 1997, 282, 223-235; David, 1998, 13, 1730-1738). These systems utilized 1α, 25-dihydroxyvitamin D₃ to stimulate stromal/osteoblastic cells to produce factors that support osteoclast formation More recent models utilize bone marrow cells cultured with soluble forms of the cytokines M-CSF (macrophage-colony stimulating factor) and a soluble form of RANKL (receptor activator of nuclear factor KB ligand) (Lacey, et al., Cell, 1988, 93, 165-176; Shevde et al., Proc. Natl. Acad. Sci. U.S.A. 2000, 97, 7829-7834). These two cytokines are now recognized as the major factors from stromal cells that support osteoclastogenesis (Takahashi, et al., Biochem. Biophys. Res. Commun., 1999, 256, 449-455). Thus, their addition to the culture medium overcomes the need for stromal cells.

“Osteoclast precursor” refers to a cell or cell structure, such as a pre-osteoclast, which is any cellular entity on the pathway of differentiation between a macrophage and a differentiated and functional osteoclast. The term osteoclast includes any osteoclast-like cell or cell structure which has differentiated fully or partially from a macrophage, and which has osteoclast character, including but not limited to positive staining for tartrate-resistant acid phosphatase (TRAP), but which is not a fully differentiated or functional osteoclast, including particularly aberrantly differentiated or non functional osteoclasts or pre-osteoclasts.

“Osteoclast culture” refers to any in vitro or ex vivo culture or system for the growth, differentiation and/or functional assessment of osteoclasts or osteoclast precursors, whether in the absence or presence of other cells or cell types, for instance, but not limited to, osteoblasts, macrophages, hematopoietic or stromal cells.

“Osteoclast function”, as used herein, refers to bone resorption and the processes required for bone resorption.

An “amount sufficient to inhibit osteoclast differentiation, formation or function” refers to the amount of the agent sufficient to block either the differentiation, the formation or the function of osteoclasts, more particularly, an amount ranging from about 0.1 nM to about 10 μM, or more preferentially from about 0.1 nM to about 5 μM, and most preferentially from about 0.1 nM to about 1 μM in vitro. In vivo amounts of an axonal guidance protein antagonist or an axonal guidance protein receptor antagonist sufficient to block either the differentiation, the formation or the function of osteoclasts may range from about 0.1 mg/Kg of body weight per day to about 200 mg/Kg of body weight per day in vivo, or more preferentially from about 1 mg/Kg to about 100 mg/Kg, and most preferentially from about 25 mg/Kg to about 50 mg/Kg of body weight per day in vivo. It is understood that the dose, when administered in vivo, may vary depending on the clinical circumstances, such as route of administration, age, weight and clinical status of the subject in which inhibition of osteoclast differentiation, formation or function is desired.

By “cell adhesion” is meant is the binding of a cell to a surface or substrate, such as an extracellular matrix or another cell. Adhesion occurs from the action of proteins, called cell adhesion molecules, or sometimes adhesins. Examples of these proteins include selectins, integrins, and cadherins. Cellular adhesion is essential in maintaining multicellular structure. Cellular adhesion can link the cytoplasm of cells and can be involved in signal transduction. Cell adhesion is also essential for the pathogenesis of infectious organisms. Dysfunction of cell-adhesion and cell-migration occurs during cancer metastasis. Cellular adhesion and traction can allow cells to migrate. Cells can form integrin mediated attachments sites called focal adhesions. Focal adhesions at the leading edge provide the necessary traction allowing the cell to pull itself forward.

By “spondyloarthropathy” or “spondyloarthrosis” is meant any joint disease of the vertebral column. As such, it is a class or category of diseases rather than a single, specific entity. It differs from spondylopathy, which is a disease of the vertebra itself. However, many conditions involve both spondylopathy and spondyloarthropathy. Spondyloarthropathy with inflammation is called spondylarthritis. In the broadest sense, the term spondyloarthropathy includes joint involvement of vertebral column from any type of joint disease, including rheumatoid arthritis and osteoarthritis, but the term is often used for a specific group of disorders with certain common features, the group often being termed specifically seronegative spondylarthropathies. They have an increased incidence of HLA-B27, as well as negative rheumatoid factor and ANA. Enthesopathy is also sometimes present in association with seronegative spondarthritides. Nonvertebral symptoms of spondyloarthropathies include asymmetric peripheral arthritis (which is distinct from rheumatoid arthritis), arthritis of the Toe IP Joints, sausage digits, Achilles tenosynovitis, plantar fasciitis, costochondritis, iritis, and mucocutaneous lesions. However, lower back pain is the most common clinical presentation of the disease; this back pain is unique because it decreases with activity.

### Ntn1 Promotes Inflammation in Inflammatory Arthritis

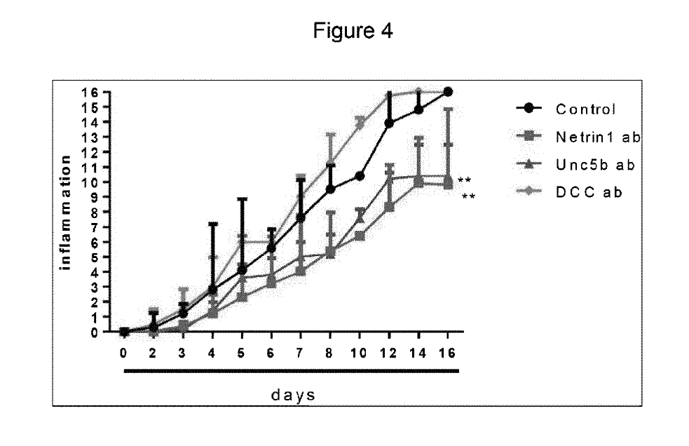

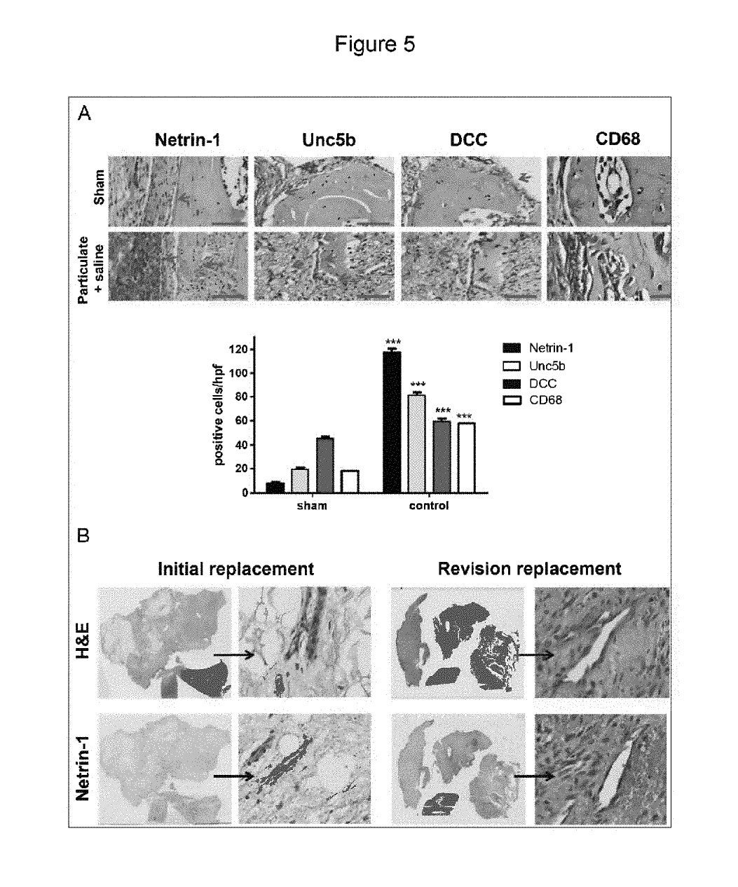

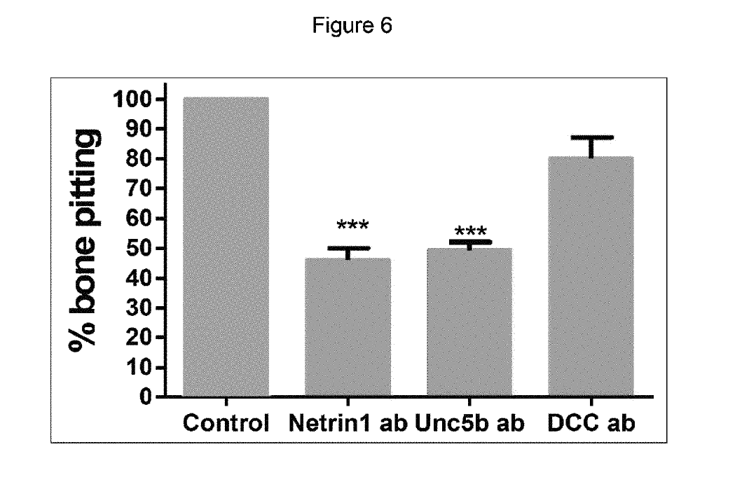

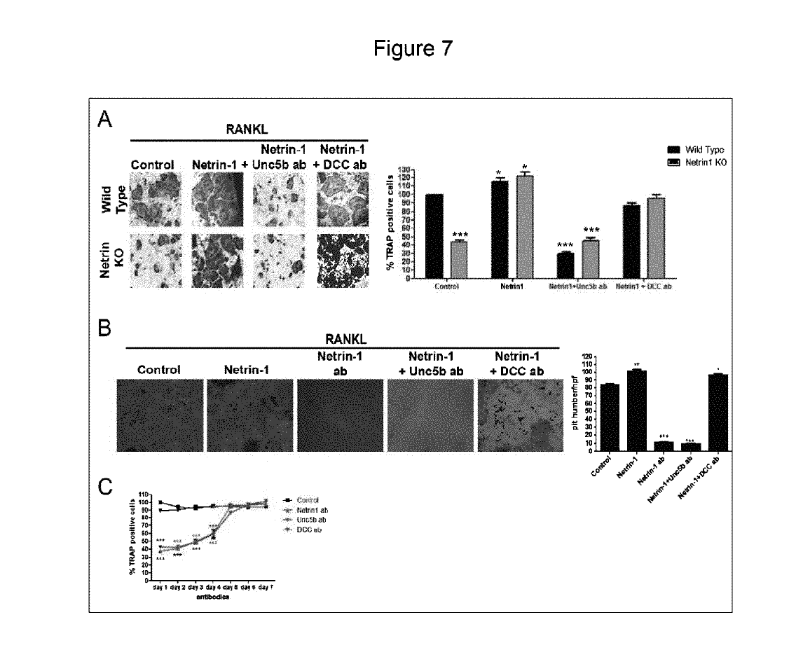

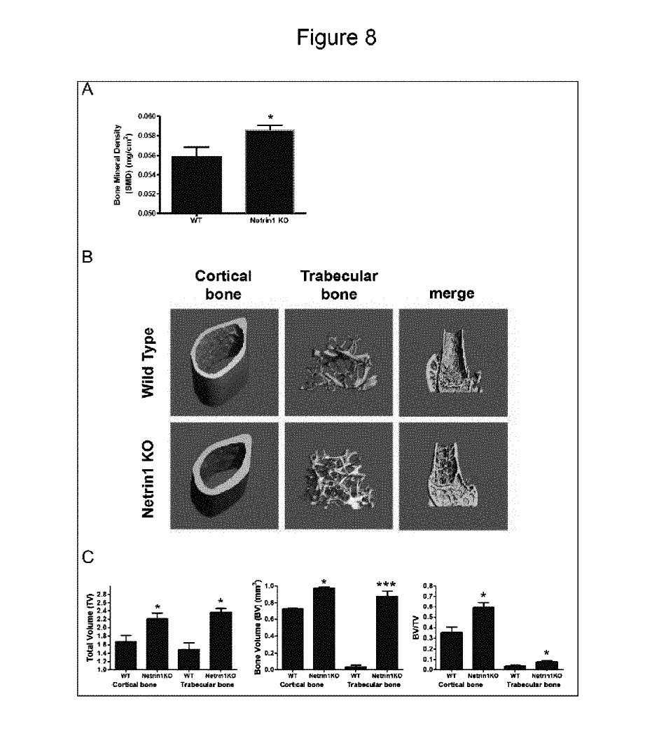

Consistent with a pro-inflammatory role of Ntn1 in arthritis, infusion of anti-Ntn1 and anti-unc5b but not anti-DCC antibodies reduce inflammation in the KBxN serum-mediated model of inflammatory arthritis (FIG. 4). High levels of Ntn1 are expressed by macrophages at sites of wear particle-induced osteolysis (FIG. 5) and anti-Ntn1 antibodies dramatically reduce macrophage accumulation at sites of wear particle-induced osteolysis (FIG. 6). Moreover, similar to their effect in inflammatory arthritis, antibodies against Ntn1 or unc5b, but not DCC, prevent the accumulation of macrophages in the periosteal area and reduce bone destruction in a model of wear particle-induced osteolysis. Interestingly, Ntn1 and unc5b are expressed by osteoclasts and that Ntn1 deficiency leads to diminished osteoclast differentiation in vitro, which can be reversed by addition of Ntn1 to cultures (FIG. 7), a finding that is consistent with an autocrine effect of Ntn1 on osteoclast differentiation. Moreover, Ntn1 deficiency in bone marrow derived cells leads to increased bone density associated with an absence of osteoclasts (FIG. 8). Antibodies to unc5b, but not DCC, inhibit osteoclast differentiation, indicating that this receptor, which is expressed by osteoclasts, is responsible for the effects of Ntn1 on osteoclast differentiation. Thus, Ntn1 should be regarded as an immune modulator rather than a strict pro- or anti-inflammatory mediator.

The present data demonstrating that inhibiting Ntn1 may successfully treat inflammatory arthritis significantly impacts the health of patients who suffer from inflammatory arthritis and bone destruction. The pathogenesis of inflammatory arthritis and bone destruction has not been fully elucidated. The demonstration that chemotropic proteins contribute to joint inflammation by preventing efflux of inflammatory cells, a previously unsuspected contribution to inflammatory arthritis, suggests novel targets for anti-inflammatory therapy in inflammatory arthritis and bone destruction and finding a novel target such as Ntn1 offers new possibilities for targeted prevention of joint destruction.

The demonstration that Ntn1 is an autocrine factor required for osteoclast function is significant. Currently, little is known about the autocrine/paracrine regulators of osteoclast differentiation and function aside from inflammatory cytokines such as TNF-α and IL-1. Moreover, although a variety of other membrane molecules such as DC-STAMP (Kukita et al., The Journal of Experimental Medicine 2004; 200(7):941-6; Yagi et al., The Journal of Experimental Medicine 2005; 202(3):345-51) and OC-STAMP (Yang et al., Journal of Cellular Physiology 2008; 215(2):497-505) regulate these cells, neither their ligands nor their mechanistic roles in osteoclastogenesis have been fully elucidated. The discovery that Ntn1 is produced during osteoclast regulation, and is required for osteoclast differentiation and function, offers a novel insight into the role of this protein in regulating bone metabolism. Moreover, the data indicate the involvement of signaling elements downstream of unc5b in macrophages, osteoclasts and T lymphocytes (that have not previously been explored in these cells) leading to novel insights into the signaling mechanisms that regulate cytoskeletal rearrangements.

The presently described methods include methods for treating inflammatory arthritis leading to joint deformity and loss of function, as well as joint prosthesis loosening due to inflammatory osteolysis. Rheumatoid arthritis, psoriatic arthritis and the spondyloarthropathies lead to osteoclast-mediated destruction of peri-articular bony structures that often necessitates joint replacement or other restorative orthopedic procedures. Patients with inflammatory arthritis are generally younger and in their productive years. Preventing joint deformity and disability is a critical need for these individuals despite the advances in therapy for inflammatory arthritis made over the past 30 years. In contrast to patients suffering from inflammatory arthritis, advancing age characterizes all of the communities affected by joint prosthesis loosening but younger men (sixth decade of life) comprise the population at greatest risk of osteolysis and prosthesis failure. Revision and replacement of joint implants often yields results that are not as satisfactory as those expected from the initial prosthesis placement. Thus, developing therapies that can halt or retard prosthesis loosening can help to prevent the need for revision and replacement of joint prostheses.

The presently described methods are based in part upon the discovery that Ntn1, an axonal guidance protein, is a regulator of osteoclast, macrophage, and T lymphocyte function that mediates its effects via ligation of a specific cell surface receptor, unc5b. The secretion, presence and function (DeGeer et al., Molecular and Cellular Biology 2013; 33(4):739-51; Briancon-Marjollet et al., Molecular and Cellular Biology 2008; 28(7):2314-23; Onel et al., Development 2004; 131(11):2587-94) of this receptor/ligand axis by osteoclasts and T lymphocytes have not been reported previously. Moreover, signaling at unc5b and DCC has been linked to cytoskeletal activation via specific guanine nucleotide exchange factors (GEFs) and dissecting the intracellular signaling events required for osteoclast differentiation under the influence of Ntn1 will shed further light on the signaling mechanisms and effectors required for osteoclast differentiation and T lymphocyte activation. Finally, and similar to other ligands, in some settings Ntn1 is anti-inflammatory whereas in other settings it plays a pro-inflammatory role. Determining the role of Ntn1 in inflammatory diseases of the joints and the bones indicates that this protein and its receptors are appropriate targets for therapy in the treatment of inflammatory conditions.

The discovery that the Ntn1/unc5b receptor/ligand complex participates in the pathogenesis of inflammatory arthritis and serves as an autocrine/paracrine regulator of osteoclast differentiation sheds new light on both the triggers for and mechanisms by which inflammatory bone damage takes place. This understanding of these systems permits design of selective and effective agents for the treatment and prevention of joint destruction.

### General Description

Selecting the compounds that interact with or bind to a protein, peptide or receptor or otherwise antagonize or agonize or stimulate the receptor may be performed in multiple ways. The compounds may first be chosen based on their structural and functional characteristics, using one of a number of approaches known in the art. For instance, homology modeling can be used to screen small molecule libraries in order to determine which molecules are candidates to interact with the receptor thereby selecting plausible targets. The compounds to be screened can include both natural and synthetic ligands. Furthermore, any desired compound may be examined for its ability to interact with or bind to the receptor.

Binding to or interaction with an axonal guidance protein or a receptor for the same may be determined by performing an assay such as, for example, a binding assay between a desired compound and a receptor. In one aspect, this is done by contacting said compound to a protein, peptide or receptor and determining its dissociation rate. Numerous possibilities for performing binding assays are well known in the art. The indication of a compound's ability to bind to a protein, peptide or receptor is determined, e.g., by a dissociation rate, and the correlation of binding activity and dissociation rates is well established in the art. For example, the assay may be performed by radio-labeling a reference compound, or other suitable radioactive marker, and incubating it with the cell bearing a receptor. Test compounds are then added to these reactions in increasing concentrations. After optimal incubation, the reference compound and receptor complexes are separated, e.g., with chromatography columns, and evaluated for bound ¹²⁵I-labeled peptide with a gamma (γ) counter. The amount of the test compound necessary to inhibit 50% of the reference compound's binding is determined. These values are then normalized to the concentration of unlabeled reference compound's binding (relative inhibitory concentration (RIC)⁻¹=concentrationₜₑₛₜ/concentration_{reference}). A small RIC⁻¹ value indicates strong relative binding, whereas a large RIC⁻¹ value indicates weak relative binding. See, for example, Latek et al., Proc. Natl. Acad. Sci. USA, 2000, 97(21): 11460-11465. A receptor agonist mimic may be computationally evaluated and designed by means of a series of steps in which chemical groups or fragments are screened and selected for their ability to associate with the individual binding pockets or interface surfaces of the protein. One skilled in the art may employ one of several methods to screen chemical groups or fragments for their ability to associate with the receptor. This process may begin by visual inspection of, for example, the protein/protein interfaces or the binding site on a computer screen based on the available crystal complex coordinates of the receptor, including a protein known to interact with selected fragments or chemical groups may then be positioned in a variety of orientations, or docked, at an individual surface of the receptor that participates in a protein/protein interface or in the binding pocket. Docking may be accomplished using software such as QUANTA and SYBYL, followed by energy minimization and molecular dynamics with standard molecular mechanics forcefields, such as CHARMM and AMBER (AMBER, version 4.0 (Kollman, University of California at San Francisco, copyright, 1994); QUANTA/CHARMM (Molecular Simulations, Inc., Burlington, Mass., copyright, 1994)). Specialized computer programs may also assist in the process of selecting fragments or chemical groups. These include: GRID (Goodford, J. Med. Chem., 1985, 28:849-857), available from Oxford University, Oxford, UK; MCSS (Miranker, et al., Proteins: Structure, Function and Genetics, 1991, 11:29-34), available from Molecular Simulations, Burlington, Mass.; AUTODOCK (Goodsell, et al., Proteins: Structure, Function, and Genetics, 1990, 8:195-202), available from Scripps Research Institute, La Jolla, Calif.; and DOCK (Kuntz et al., J. Mol. Biol., 1982, 161:269-288), available from University of California, San Francisco, Calif. Once suitable chemical groups or fragments that bind to an axonal guidance protein or receptor thereof have been selected, they can be assembled into a single compound or agonist. Assembly may proceed by visual inspection of the relationship of the fragments to each other in the three-dimensional image displayed on a computer screen in relation to the structure coordinates thereof. This would be followed by manual model building using software such as QUANTA or SYBYL. Useful programs to aid one of skill in the art in connecting the individual chemical groups or fragments include: CAVEAT (Bartlett et al., 1989, ‘CAVEAT: A Program to Facilitate the Structure-Derived Design of Biologically Active Molecules’. In Molecular Recognition in Chemical and Biological Problems', Special Pub., Royal Chem. Soc. 78:182-196), available from the University of California, Berkeley, Calif.; 3D Database systems such as MACCS-3D (MDL Information Systems, San Leandro, Calif.). This area is reviewed in Martin, J. Med. Chem., 1992, 35:2145-2154); and HOOK (available from Molecular Simulations, Burlington, Mass.). Instead of proceeding to build an antagonist to an axonal guidance protein or receptor thereof mimic, in a step-wise fashion one fragment or chemical group at a time, as described above, such compounds may be designed as a whole or ‘de novo’ using either an empty binding site or the surface of a protein that participates in protein/protein interactions or optionally including some portion(s) of a known activator(s). These methods include: LUDI (Bohm, J. Comp. Aid. Molec. Design 1992, 6:61-78), available from Molecular Simulations, Inc., San Diego, Calif.; LEGEND (Nishibata et al., Tetrahedron, 1991, 47:8985), available from Molecular Simulations, Burlington, Mass.; and LeapFrog (available from Tripos, Inc., St. Louis, Mo.). Other molecular modeling techniques may also be employed in accordance with this invention. See, e.g., Cohen et al., J. Med. Chem., 1990, 33:883-894. See also, Navia, et al., Current Opinions in Structural Biology, 1992, 2:202-210.

Once a compound has been designed by the above methods, the efficiency with which that compound may bind to or interact with the receptor protein may be tested and optimized by computational evaluation. Agonists may interact with the receptor in more than one conformation that is similar in overall binding energy. In those cases, the deformation energy of binding is taken to be the difference between the energy of the free compound and the average energy of the conformations observed when the inhibitor binds to the receptor protein.

A compound selected for binding to the receptor protein may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the compound and the receptor protein when the mimic is bound to it preferably make a neutral or favorable contribution to the enthalpy of binding. Specific computer software is available in the art to evaluate compound deformation energy and electrostatic interaction. Examples of programs designed for such uses include: Gaussian 92, revision C (Frisch, Gaussian, Inc., Pittsburgh, Pa. copyright 1992); AMBER, version 4.0 (Kollman, University of California at San Francisco, copyright 1994); QUANTA/CHARMM (Molecular Simulations, Inc., Burlington, Mass., copyright 1994); and Insight II/Discover (Biosym Technologies Inc., San Diego, Calif., copyright 1994). These programs may be implemented, for instance, using a computer workstation, as are well-known in the art. Other hardware systems and software packages will be known to those skilled in the art.

Once a receptor modulating compound has been optimally designed, for example as described above, substitutions may then be made in some of its atoms or chemical groups in order to improve or modify its binding properties, or its pharmaceutical properties such as stability or toxicity. Generally, initial substitutions are conservative, i.e., the replacement group will have approximately the same size, shape, hydrophobicity and charge as the original group. Substitutions known in the art to alter conformation should be avoided. Such altered chemical compounds may then be analyzed for efficiency of binding to the receptor by the same computer methods described in detail above.

### Candidate Compounds and Agents

Examples of agents, candidate compounds or test compounds include, but are not limited to, nucleic acids (e.g., DNA and RNA), carbohydrates, lipids, proteins, peptides, peptidomimetics, small molecules and other drugs. In one preferred aspect, agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des., 1997, 12:145; U.S. Pat. No. 5,738,996 and U.S. Pat. No. 5,807,683).

Phage display libraries may be used to screen potential ligands or axonal guidance protein modulators. Their usefulness lies in the ability to screen, for example, a library displaying a large number of different compounds. For use of phage display libraries in a screening process, see, for instance, Kay, et al., Methods, 2001, 240-246. An exemplary scheme for using phage display libraries to identify compounds that bind or interact with an axonal guidance protein or its receptor may be described as follows: initially, an aliquot of the library is introduced into microtiter plate wells that have previously been coated with target protein, e.g. guidance protein or its receptor. After incubation (e.g., 2 hours), the nonbinding phage are washed away, and the bound phage are recovered by denaturing or destroying the target with exposure to harsh conditions such as, for instance pH 2, but leaving the phage intact. After transferring the phage to another tube, the conditions are neutralized, followed by infection of bacteria with the phage and production of more phage particles. The amplified phage is then rescreened to complete one cycle of affinity selection. After three or more rounds of screening, the phage is plated out such that there are individual plaques that can be further analyzed. For example, the conformation of binding activity of affinity-purified phage for the guidance protein or its receptor may be obtained by performing ELISAs. One skilled in the art can easily perform these experiments. In one aspect, an guidance protein or its receptor used for any of the assays may be a recombinant guidance protein or its receptor, or a fusion protein, an analog, derivative, or mimic thereof

Examples of methods for the synthesis of molecular libraries can be found in the art, for example in: DeWitt et al., Proc. Natl. Acad. Sci. USA, 1993, 90:6909; Erb et al., Proc. Natl. Acad. Sci. USA, 1994, 91:11422; Zuckermann et al., J. Med. Chem., 1994, 37:2678; Cho et al., Science, 1993, 261:1303; Carrell et al., Angew. Chem. Int. Ed. Engl., 1994, 33:2059; Carell et al., Angew. Chem. Int. Ed. Engl., 1994, 33:2061; and Gallop et al., J. Med. Chem., 1994, 37:1233.

Libraries of compounds may be presented, e.g., in solution (Houghten, Bio/Techniques, 1992, 13:412-421), or on beads (Lam, Nature, 1991, 354:82-84), chips (Fodor, Nature, 1993, 364:555-556), bacteria (U.S. Pat. No. 5,223,409), spores (U.S. Pat. Nos. 5,571,698; 5,403,484; and 5,223,409), plasmids (Cull et al., Proc. Natl. Acad. Sci. USA 1992, 89:1865-1869) or phage (Scott, et al., Science, 1990, 249:386-390; Devlin, Science, 1990, 249:404-406; Cwirla et al., Proc. Natl. Acad. Sci. USA, 1990, 87:6378-6382; and Felici, J. Mol. Biol., 1991, 222:301-310).

The methods of screening compounds may also include the specific identification or characterization of such compounds, whose effect on bone resorption was determined by the methods described above. If the identity of the compound is known from the start of the experiment, no additional assays are needed to determine its identity. However, if the screening for compounds that modulate the protein, peptide or receptor is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled artisan (e.g. the neogenesis website). Neogenesis' ALIS (automated ligand identification system) spectral search engine and data analysis software allow for a highly specific identification of a ligand structure based on the exact mass of the ligand. One skilled in the art can also readily perform mass spectrometry experiments to determine the identity of the compound.

Antibodies, including polyclonal and monoclonal antibodies, for instance anti-A_{2A} receptor antibodies and neutralizing antibodies may be useful as compounds to modulate osteoclast differentiation and/or function. These antibodies are available from such vendors as Upstate Biologicals, Santa Cruz, or they made be prepared using standard procedures for preparation of polyclonal or monoclonal antibodies known to those skilled in the art. Also, antibodies including both polyclonal and monoclonal antibodies, and drugs that modulate the activity of the axonal guidance protein or receptor thereof and/or its subunits may possess certain diagnostic applications and may for example, be utilized for the purpose of detecting and/or measuring conditions such as inflammation. The axonal guidance protein or its receptor or its subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or act as agonists for the activities of the A_{2A} receptor may be discovered or synthesized, and may be used in diagnostic and/or therapeutic protocols.

### Therapeutic and Prophylactic Compositions and Their Use

Candidates for therapy with the agents identified by the methods described herein are patients either suffering from inflammation, inflammatory arthritis or bone resorption or at risk for doing so.

The invention provides methods of treatment featuring administering to a subject an effective amount of an agent of the invention. The compound is preferably substantially purified (e.g., substantially free from substances that limit its effect or produce undesired side-effects). The subject is preferably an animal, including but not limited to animals such as monkeys, cows, pigs, horses, chickens, cats, dogs, etc., and is preferably a mammal, and most preferably human. In one specific embodiment, a non-human mammal is the subject. In another specific embodiment, a human mammal is the subject. Accordingly, the agents identified by the methods described herein may be formulated as pharmaceutical compositions to be used for prophylaxis or therapeutic use to treat these patients.

Various delivery systems are known and can be used to administer a compound of the invention, e.g., encapsulation in liposomes, microparticles, or microcapsules. Methods of introduction can be enteral or parenteral and include but are not limited to intradermal, intramuscular, intraperitoneal, intravenous, subcutaneous, intranasal, epidural, topical and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compositions of the invention locally to the area in need of treatment.

Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, the compound can be delivered in a vesicle, in particular a liposome (Langer, Science, 1990, 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327)

In yet another embodiment, the compound can be delivered in a controlled or sustained release system. In one embodiment, a pump may be used (see Langer, supra; Sefton CRC Crit. Ref. Biomed. Eng. 1987, 14:201; Buchwald et al., Surgery, 1980, 88:507; Saudek et al., N Engl. J. Med. 1989, 321:574). In another embodiment, polymeric materials can be used (See, Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., Macromol. Sci. Rev. Macromol. Chem. 1983, 23:61; Levy et al., Science, 1985, 228:190; During et al. Ann. Neurol., 1989, 25:351; Howard et al., J. Neurosurg., 1989, 71:105). In yet another embodiment, a controlled release system can be placed in proximity of the therapeutic target, i.e., the subject bone or prosthesis, thus requiring only a fraction of the systemic dose (see, e.g., Goodson, in Medical Applications of Controlled Release (1984) supra, vol. 2, pp. 115-138). Other suitable controlled release systems are discussed in the review by Langer, Science, 1990, 249:1527-1533.

The present invention further contemplates therapeutic compositions useful in practicing the therapeutic methods of this invention. A subject therapeutic composition includes, in admixture, a pharmaceutically acceptable excipient (carrier).

Effects of the compounds or agents of the invention can first be tested for their ability to inhibit or antagonize an axonal guidance protein such as netrin-1 or a receptor of the same, such as unc5b, using standard techniques known in the art. More particularly, the selectivity of the compounds for the receptor can be assessed using radioligand binding assays whereby a test or candidate compound can be assayed for its ability to bind to a cell having or expressing the receptor. Cells can be transfected with the nucleic acid encoding the various receptors and competitive binding assays with radiolabeled ligands run to evaluate the specificity of the particular candidate compounds.

The present compounds or agents that modulate the axonal guidance protein or its receptor can be used as the sole active agents, or can be used in combination with one or more other active ingredients. In particular, combination therapy using one or more other anti-inflammatory agents is contemplated. These agents are known in the art, and can be selected from anti-inflammatory compounds, bisophosphonates, soluble RANK, and bone morphogenetic proteins, for instance.

When contemplating combination therapy with a modulator, inhibitor or antagonist of an axonal guidance protein or its receptor and one or more of the above-noted agents, it is important to assess clinical safety by methods known to those skilled in the art. Appropriate dose titration may be necessary when certain groups of compounds are contemplated for use together.

The compounds or compositions of the invention may be combined for administration with or embedded in polymeric carrier(s), biodegradable or biomimetic matrices or in a scaffold. The carrier, matrix or scaffold may be of any material that will allow the composition to be incorporated and expressed and will be compatible with the addition of cells or in the presence of cells. Preferably, the carrier matrix or scaffold is predominantly non-immunogenic and is biodegradable. Examples of biodegradable materials include, but are not limited to, polyglycolic acid (PGA), polylactic acid (PLA), hyaluronic acid, catgut suture material, gelatin, cellulose, nitrocellulose, collagen, albumin, fibrin, alginate, cotton, or other naturally-occurring biodegradable materials. It may be preferable to sterilize the matrix or scaffold material prior to administration or implantation, e.g., by treating it with ethylene oxide or by gamma irradiation or irradiation with an electron beam. In addition, a number of other materials may be used to form the scaffold or framework structure, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), polymers of hydroxy acids such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof. Matrices suitable include a polymeric mesh or sponge and a polymeric hydrogel. In the preferred embodiment, the matrix is biodegradable over a time period of less than a year, more preferably less than six months, most preferably over two to ten weeks. The polymer composition, as well as method of manufacture, can be used to determine the rate of degradation. For example, mixing increasing amounts of polylactic acid with polyglycolic acid decreases the degradation time. Meshes of polyglycolic acid that can be used can be obtained commercially, for instance, from surgical supply companies (e.g., Ethicon, N.J.). A hydrogel is defined as a substance formed when an organic polymer (natural or synthetic) is cross-linked via covalent, ionic, or hydrogen bonds to create a three-dimensional open-lattice structure which entraps water molecules to form a gel. In general, these polymers are at least partially soluble in aqueous solutions, such as water, buffered salt solutions, or aqueous alcohol solutions that have charged side groups, or a monovalent ionic salt thereof

For use in treating animal subjects, the compositions of the invention can be formulated as pharmaceutical or veterinary compositions. Depending on the subject to be treated, the mode of administration, and the type of treatment desired, e.g., prevention, prophylaxis, therapy; the compositions are formulated in ways consonant with these parameters. A summary of such techniques is found in Remington's Pharmaceutical Sciences, latest edition, Mack Publishing Co., Easton, Pa.

The preparation of therapeutic compositions containing small organic molecules polypeptides, analogs or active fragments as active ingredients is well understood in the art. The compositions of the present invention may be administered parenterally, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Formulations may be prepared in a manner suitable for systemic administration or for topical or local administration. Systemic formulations include, but are not limited to those designed for injection (e.g., intramuscular, intravenous or subcutaneous injection) or may be prepared for transdermal, transmucosal, nasal, or oral administration. Such compositions may be prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. The formulation will generally include a diluent as well as, in some cases, adjuvants, buffers, preservatives and the like. In addition, if desired, the composition can contain minor amounts of auxiliary substances such as wetting or emulsifying agents, pH buffering agents which enhance the effectiveness of the active ingredient.

A small organic molecule/compound, a polypeptide, an analog or active fragment thereof can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like. For oral administration, the compositions can be administered also in liposomal compositions or as microemulsions. Suitable forms include syrups, capsules, tablets, as is understood in the art.

The compositions of the present invention may also be administered locally to sites in subjects, both human and other vertebrates, such as domestic animals, rodents and livestock, using a variety of techniques known to those skilled in the art. For example, these may include sprays, lotions, gels or other vehicles such as alcohols, polyglycols, esters, oils and silicones.

The administration of the compositions of the present invention may be pharmacokinetically and pharmacodynamically controlled by calibrating various parameters of administration, including the frequency, dosage, duration mode and route of administration. Variations in the dosage, duration and mode of administration may also be manipulated to produce the activity required.

The therapeutic compositions are conventionally administered in the form of a unit dose, for instance intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the agent selected for treating the subject, the dosage formulation, and in a therapeutically effective amount. If one desires to achieve the desired effect in vitro, the effective amounts may range from about 0.1 nM to about 10 μM, more preferably about 0.1 nM to about 5 μM, and most preferably from about 0.1 nM to about 1 nM. The desired effect refers to the effect of the agent on reducing inflammation and/or reducing or inhibiting osteoclast differentiation or stimulation, and reducing or inhibiting bone resorption. Moreover, the quantity of the inhibitor or modulator of the axonal guidance protein or its receptor to be administered depends on the subject to be treated, and degree of inhibition or antagonism of the axonal guidance protein or its receptor desired or the extent or severity of bone resorption or inflammation or inflammatory arthritis. Precise amounts of active ingredient required to be administered depend on the judgment of the practitioner and are peculiar to each individual. However, suitable dosages to achieve the desired therapeutic effect in vivo may range from about 0.1 mg/kg body weight per day to about 200 mg/kg body weight per day, or from about 1.0 mg/kg body weight per day to about 100 mg/kg body weight per day, preferably about 25 mg/kg body weight per day to about 50 mg/kg body weight per day. In a particular embodiment, the term “about” means within 20%, preferably within 10%, and more preferably within 5%. The preferred dose will depend on the route of administration. However, dosage levels are highly dependent on the nature of the disease or situation, the condition of the subject, the judgment of the practitioner, and the frequency and mode of administration. If the oral route is employed, the absorption of the substance will be a factor effecting bioavailability. A low absorption will have the effect that in the gastro-intestinal tract higher concentrations, and thus higher dosages, will be necessary. Suitable regimes for initial administration and further administration are also variable, but are typified by an initial administration followed by repeated doses at one or more hour intervals by a subsequent injection or other administration. Alternatively, continuous intravenous infusion sufficient to maintain desired concentrations, e.g. in the blood, are contemplated. The composition may be administered as a single dose multiple doses or over an established period of time in an infusion.

It will be understood that the appropriate dosage of the substance should suitably be assessed by performing animal model tests, where the effective dose level (e.g., ED₅₀) and the toxic dose level (e.g. TD₅₀) as well as the lethal dose level (e.g. LD₅₀ or LD₁₀) are established in suitable and acceptable animal models. Further, if a substance has proven efficient in such animal tests, controlled clinical trials should be performed.

The compounds or compositions of the present invention may be modified or formulated for administration at the site of pathology. Such modification may include, for instance, formulation which facilitate or prolong the half-life of the compound or composition, particularly in the environment. Additionally, such modification may include the formulation of a compound or composition to include a targeting protein or sequence which facilitates or enhances the uptake of the compound/composition to bone or bone precursor cells. In a particular embodiment, such modification results in the preferential targeting of the compound to the site of arthritis, joints, bone or bone precursor cells versus other locations or cells. In one embodiment, a tetracycline, tetracycline family or bisphosphonate may be utilized to target the compound or composition of the present invention to bone or bone cells, including osteoclasts and osteoclast precursors. Novel heterocycles as bone targeting compounds are disclosed in U.S. Patent Publication No. 2002/0103161 A, which is incorporated herein by reference in its entirety.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Sterile injectable forms of the compositions may be aqueous or oleaginous suspensions. The suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered once a day or on an “as needed” basis.

The pharmaceutical compositions may be orally administered in any orally acceptable dosage form including, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically. Topical application can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The invention also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### Effective Doses

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from cell culture assays and animal studies can be used in formulating a dose range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to optimize efficacious doses for administration to humans. Plasma levels can be measured by any technique known in the art, for example, by high performance liquid chromatography.

In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Normal dose ranges used for particular therapeutic agents employed for specific diseases can be found in the Physicians' Desk Reference 54^{th} Edition (2000).

### EXAMPLES

The following examples are set forth to provide those of ordinary skill in the art with a description of how to make and use the methods and compositions of the invention, and are not intended to limit the scope thereof. Efforts have been made to insure accuracy of numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Antibodies to Ntn1 and unc5b Diminish Arthritis Severity

Mice were injected intraperitoneally with 350 μl of serum from KBxN arthritic mice and anti-ntn1, -unc5b or -DCC antibodies (100 ml) or saline. Severity was assessed and each point represents the mean (±SD) of severity of 10 (Control and anti-ntn1 antibody treated) or 5 mice. The maximum severity score is 16 for each mouse (Inflammation is scored 0-4 on 4 paws) ***p<0.001, ANOVA with repeated measures. All of the antibodies used were monoclonal. The results indicate that antibodies to Ntn1 and unc5b, but not DCC diminish arthritis severity in the KBxN serum-induced arthritis model.

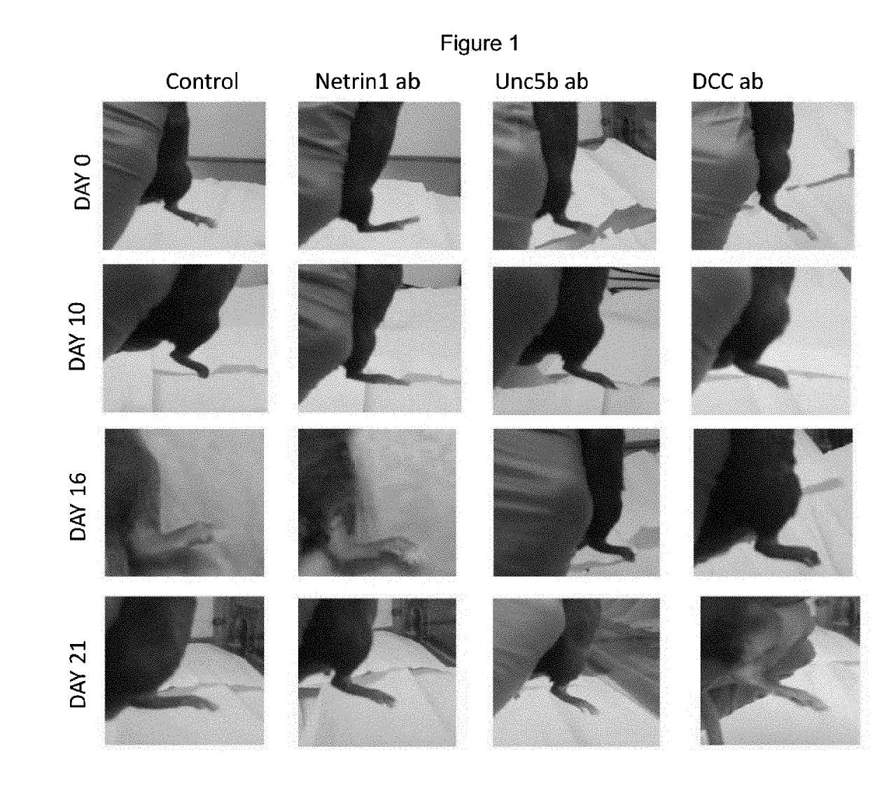

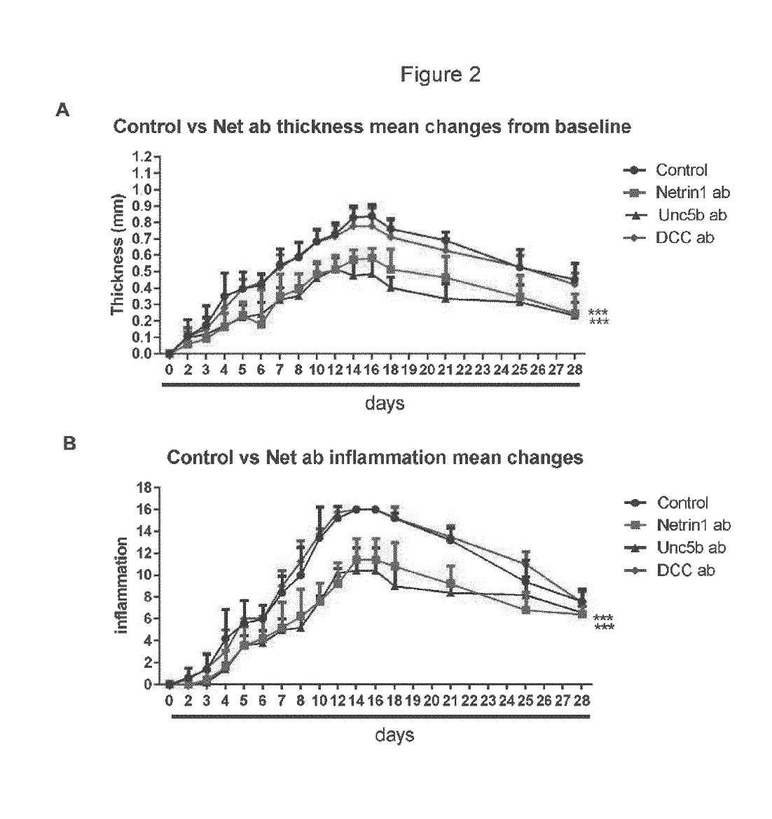

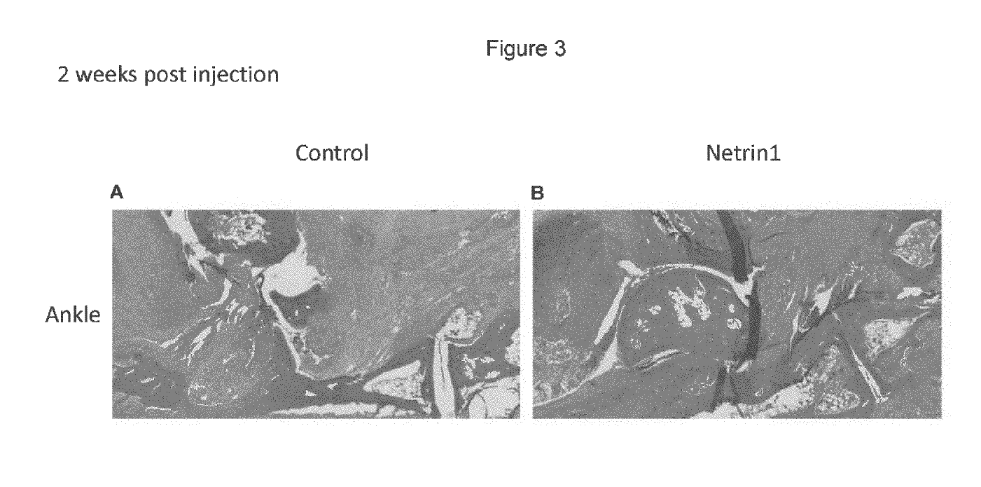

FIG. 1 demonstrates morphologically that antibodies to Ntn1 and unc5b, but not DCC, diminish arthritis severity in the KBxN serum-induced arthritis model by reducing paw thickness. FIG. 2 demonstrates that antibodies to Ntn1 and unc5b, but not DCC, diminish arthritis severity in the KBxN serum-induced arthritis model by reducing or slowing inflammation in addition to reducing paw thickness. FIG. 3 demonstrates the results of anti-ntn1 monoclonal antibodies histologically at the site of pathology.

### Example 2

### Ntn1 Plays A Critical Role in Inflammation And Bone Destruction During Inflammatory Arthritis and Osteolysis

### Background

Ntn1 has generally been reported to have anti-inflammatory properties in a number of different settings, including inflammatory bowel disease, pancreatitis, peritonitis and pulmonary inflammation (Aherne et al., Gut 2012; 61(5):695-705; Chen et al., PloS one 2012; 7(9):e46201; Grenz et al., PloS one 2011; 6(5):e14812; Han et al., Investigative Ophthalmology & Visual Science 2012; 53(3):1285-95; Ly et al., Proceedings of the National Academy of Sciences of the United States of America 2005; 102(41):14729-34; Mirakaj et al, Journal of Immunology 2011; 186(1):549-55; Mirakaj et al., American Journal of Respiratory and Critical Care Medicine 2010; 181(8):815-24; Schubert et al., International Journal of Immunopathology and Pharmacology 2009; 22(3):715-22; Tadagavadi et al., Journal of Immunology 2010;185(6):3750-8; Wang et al., American Journal of Physiology Renal Physiology 2008; 294(4):F739-47). Nonetheless, Ntn1 plays a critical role in atherogenesis and obesity-related insulin resistance by inhibiting the efflux of inflammatory macrophages from affected tissues, e.g. atherosclerotic plaques and visceral adipose tissue (van Gils et al., Nature immunology 2012; 13(2):136-43, Ramkhelawon et al., Arteriosclerosis, Thrombosis, and Vascular Biology 2013; 33(6):1180-8).

Wear particle-induced osteolysis is an inflammatory lesion in which metallic and ultra-high molecular weight particles derived from prostheses provoke an inflammatory lesion leading to osteolysis and bone loss (Mediero et al., Science Translational Medicine 2012; 4(135):135ra65). CD68⁺macrophages in the inflammatory infiltrate (Mediero et al., Science Translational Medicine 2012; 4(135):135ra65), the majority of the cells present, express Ntn1, as do the osteoclasts in the underlying bone (FIG. 5). Moreover, Ntn1 and unc5b antibodies diminish inflammatory cell accumulation at sites of wear particle-induced inflammation (FIG. 6). Similarly, in the KBxN arthritis model, anti-Ntn1 and anti-unc5b antibodies diminish the intensity of arthritis that develops in mice infused with serum from KBxN mice (FIG. 4). Ntn1 likely promotes inflammation by blocking egress of macrophages (and possibly other cell types) from the inflamed site, and/or enhancing macrophage inflammatory responses, as observed in the atherosclerotic plaque and adipose tissue (van Gils et al., Nature immunology 2012; 13(2):136-43; Ramkhelawon et al., Nature Medicine 2014; 20(4):377-84). Moreover, Ntn1 is required for inflammatory bone destruction via direct regulation of osteoclast differentiation and function.

The current understanding of the pathogenesis of rheumatoid arthritis and other types of inflammatory arthritis emphasize the role of cytokines, chemoattractants and adhesion molecules in maintaining inflammation in the synovium. The role of factors that prevent efflux of inflammatory cells from the synovium in the pathogenesis of persistence of inflammatory arthritis has not been examined. Moreover, the role of agents that retard the emigration of inflammatory cells from the synovium in the destruction of the underlying bone and cartilage has not been examined. Bone destruction is a central characteristic of rheumatoid arthritis and other forms of inflammatory arthritis. Because synovial fibroblasts from patients with rheumatoid arthritis also produce Ntn1 (Schubert et al., International Journal of Immunopathology and Pharmacology 2009; 22(3):715-22), Ntn1 plays a role in the pathogenesis of rheumatoid arthritis. Nonetheless, as described above for wear particle-induced osteolysis, Ntn1 may be anti-inflammatory in the setting of inflammatory arthritis, as reported for other inflamed sites (Aherne et al., Gut 2012; 61(5):695-705; Chen et al., PloS one 2012; 7(9):e46201; Grenz et al., PloS one 2011; 6(5):e14812; Han et al., Investigative Ophthalmology & Visual Science 2012; 53(3):1285-95; Ly et al., Proceedings of the National Academy of Sciences of the United States of America 2005; 102(41):14729-34; Mirakaj et al, Journal of Immunology 2011; 186(1):549-55; Mirakaj et al., American Journal of Respiratory and Critical Care Medicine 2010; 181(8):815-24; Schubert et al., International Journal of Immunopathology and Pharmacology 2009; 22(3):715-22; Tadagavadi et al., Journal of Immunology 2010;185(6):3750-8; Wang et al., American Journal of Physiology Renal Physiology 2008; 294(4):F739-47) or may exacerbate inflammation by inhibiting egress of inflammatory cells from the inflamed joint, as in the atherosclerotic plaque (van Gils et al., Nature immunology 2012; 13(2):136-43). Further understanding of the role of Ntn1 in the pathogenesis of inflammatory arthritis and peri-articular bone destruction is desirable.

### Materials and Methods

Murine models of inflammatory arthritis and bone destruction will be examined to determine whether Ntn1 plays a role in the pathogenesis of bone destruction in the setting of inflammatory arthritis and wear particle-induced osteolysis. The effects of antibodies to Ntn1 and its receptors (unc5b and DCC) will be compared to Ntn1 deletion in the hematopoietic compartment of mice (Radiation chimeras as shown in FIG. 8 and (van Gils et al., Arteriosclerosis, Thrombosis, and Vascular Biology 2013; 33(5):911-9)). In addition the role of selective deletion of Ntn1 (breed floxed Ntn1) or its receptor unc5b (breed floxed unc5b) will be examined in osteoclasts (cathepsinK-cre), osteoblasts (Co12.3-cre), macrophages (lysM-cre), and T cells (lck-cre) on development of inflammatory arthritis and associated bone destruction to better appreciate how Ntn1 affects arthritis severity and bone destruction.

1. KBxN Arthritis Model

The role of Ntn1 in inflammatory arthritis and bone destruction will be examined by studying the effects of Ntn1, unc5b and DCC blocking antibodies, Ntn1 deficiency (in mice with either Ntn1 -null bone marrow or cell-specific deletion of Ntn1) in a murine model of inflammatory arthritis. In this model mice are injected with serum from KBxN mice that form antibodies to glucose 6-phosphate isomerase following which they develop inflammatory arthritis (Matsumoto et al., Science 1999; 286(5445):1732-5; Kyburz et al., Springer Seminars in Immunopathology 2003; 25(1):79-90; Monach et al., Current Protocols in Immunology 2008; Chapter 15:Unit 15 22). This model is advantageous as it is not dependent on genetic background of the mice that develop arthritis. Thus, the severity and bony destruction in mice lacking Ntn1 can be assessed quantitatively despite breeding of these mice on C57BL6 background. Severity of arthritis will be assessed using a standard severity score based on erythema and swelling of knees and feet in mice (0 is unaffected, 4 is maximal erythema and swelling; a maximum score of 16 (4×4 legs) per mouse). Bone destruction will be assessed by micro CT and histology/immunohistology. Results clearly indicate a role for Ntn1 in inflammatory arthritis (FIG. 4). Reversal of Ntn1 deficiency will be tested by administration of exogenous Ntn1 to the mice in selective KO mice. Similarly antibodies (murine monoclonal) to unc5b and DCC will be administered to confirm that the same receptors are active in vivo as we have seen in vitro.

2. Collagen-Induced Arthritis Model

Because the KBxN model of inflammatory arthritis described above is a passive form of arthritis that does not require the involvement of the adaptive immune system, the role of Ntn1 in the pathogenesis of inflammatory arthritis will be studied in a second model which is more dependent on adaptive immunity: the collagen-induced arthritis (CIA) model. The arthritis following immunization with collagen is more robust in DBA/DBA mice, and thus initial studies will be performed using antibodies to Ntn1, unc5b and DCC (and isotype controls). If initial studies reveal involvement of Ntn1 and its receptors in the development of arthritis in this model, the effects of cell-selective Ntn1 deletion in the mice described above will be evaluated realizing that the incidence of arthritis in mice with a C57B/6 background is significantly less (approximately 50% in most reports vs. 80-90% for DBA/DBA mice).

3. Wear Particle-Induced Osteolysis Model

In recent studies the role of adenosine receptor agonists in diminishing wear particle-induced bone resorption in a murine model were determined (Mediero et al., Science Translational Medicine 2012; 4(135):135ra65). In this model wear particles are placed in apposition to calvaria, and the area of bone destruction is digitally determined in micro CT images of the affected skull. In these experiments, application of an adenosine A_{2A} receptor agonist has been shown to inhibit bone destruction, confirmed by histology and immunohistology. In more recent published experiments it was shown that, in addition to diminishing inflammatory infiltrate and osteoclasts at the site, there is an increase in osteoblasts associated with increased bone formation, as shown by fluorescence imaging of mice injected with a fluorescent dye that binds to hydroxyapatite in newly formed bone (Xenolight Bone Probe 680 (Mediero et al., Arthr Rheum 2014; In Press). Wear particle-induced osteolysis will be studied in this mouse model by comparing bone destruction in mice transplanted with either wild type or Ntn1 deficient bone marrow, and in mice with selective deletion of Ntn1 in osteoclasts or macrophages. Inflammation and bone destruction will be measured by histologic and immunohistologic analyses and cytokine synthesis and release will be determined by multiplex Luminex assay, as previously described (Mediero et al., Science Translational Medicine 2012; 4(135):135ra65). Reversal of Ntn1 deficiency will be tested by administration of exogenous Ntn1 to the mice. Likewise antibodies to unc5b and DCC will be administered to confirm that the same receptors are active in vivo, as demonstrated in vitro.

### Results

There are three potential outcomes in each model to be studied. First, Ntn1 deficiency or blockade may lead to diminished inflammation and diminished bone destruction in the arthritis and wear-particle-induced osteolysis models of inflammation. Although many prior studies have shown anti-inflammatory actions of Ntn1 ((Aherne et al., Gut 2012; 61(5):695-705; Chen et al., PloS one 2012; 7(9):e46201; Grenz et al., PloS one 2011; 6(5):e14812; Han et al., Investigative Ophthalmology & Visual Science 2012; 53(3):1285-95; Ly et al., Proceedings of the National Academy of Sciences of the United States of America 2005; 102(41):14729-34; Mirakaj et al, Journal of Immunology 2011; 186(1):549-55; Mirakaj et al., American Journal of Respiratory and Critical Care Medicine 2010; 181(8):815-24; Schubert et al., International Journal of Immunopathology and Pharmacology 2009; 22(3):715-22; Tadagavadi et al., Journal of Immunology 2010;185(6):3750-8; Wang et al., American Journal of Physiology Renal Physiology 2008; 294(4):F739-47)), the recent reports (van Gils et al., Nature immunology 2012; 13(2):136-43; Ramkhelawon et al., Nature Medicine 2014; 20(4):377-84) demonstrate that by inhibiting macrophage efflux from an inflamed site (atherosclerotic plaque or adipose tissue) Ntn1 may promote inflammation. Since macrophages play a central role in synovial and periarticular damage in inflammatory arthritis, Ntn1-mediated inhibition of their efflux may play a role in tissue damage in arthritis, and deletion of Ntn1 may diminish accumulation of macrophages and expression of the cytokines (TNF-α, IL-1) that promote osteoclast function. The inflammatory infiltrate in wear particle-induced osteolysis is also composed almost exclusively of macrophages (CD68⁺) (Mediero et al., Science Translational Medicine 2012; 4(135):135ra65), which can stimulate osteoclast function via secretion of TNFα and other cytokines (Mediero et al., Science Translational Medicine 2012; 4(135):135ra65). In both of these models, osteoclasts mediate the bony destruction and Ntn1 deficiency leads to reduced or absent osteoclast differentiation and function. Second, Ntn1 deficiency may lead to diminished bony destruction, but inflammation is unaffected or even enhanced. In this scenario the retention of macrophages at the inflamed site is not critical for persistent inflammation. The absence of osteoclast differentiation in the setting of Ntn1 deficiency should diminish bony destruction, but absence of Ntn1-mediated anti-inflammatory effects would permit or exacerbate persistent inflammation. Third, Ntn1 deficiency may lead to unchanged or increased inflammation and bony destruction is unaffected by loss of Ntn1 expression. In this scenario the loss of Ntn1 does not affect persistence of macrophages at the inflamed sites, loss of the anti-inflammatory effect of Ntn1 allows or exaggerates inflammation and alternative stimulation (e.g. by increased TNF-α production) permits or exacerbates bone destruction by osteoclasts. In any of these scenarios the role of Ntn1 will be central to the outcome: enhanced or diminished inflammation associated with diminished or persistent bony destruction.

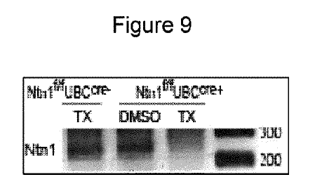

In further experiments, whether targeted deletion of Ntn1, induced after inflammatory arthritis is established, can reverse macrophage accumulation and T cell recruitment will be examined. To test this, bone marrow chimeric Ntn1^{fl/fl}UBCcre/ERT2 (or control WTUBCcre/ERT2) mice treated with serum from KBxN mice, and in which hematopoietic Ntn1 can be inducibly deleted by tamoxifen as shown in FIG. 9, will be used. After 16 days, severity of arthritis and bone destruction will be measured, and effects on macrophage and T cell accumulation, survival and inflammatory gene expression will be measured. This will demonstrate the value of Ntn1 as a therapeutic target in inflammatory arthritis.

In a complementary approach, the ability of T cells and macrophages lacking Ntn1 receptors to migrate to the inflamed joint or site of wear particle deposition will be evaluated. Specifically, the KBxN serum-treated mice will be injected with fluorescently tagged cells where either unc5b or DCC (control) genes will be depleted after introduction of inhibitory oligonucleotides. The ability of these cells to migrate from the peripheral circulation and to inhabit the synovial/subperiosteal compartment will be documented by histological studies. These experiments should differentiate the roles of Ntn1 in either migration to or from articular compartments.

### Example 3

### Ntn1 and its Receptors Regulate Immune Cell and Osteoclast Function

### Background

### Ntn1 Regulates Activated Macrophage Accumulation at Inflamed Sites.

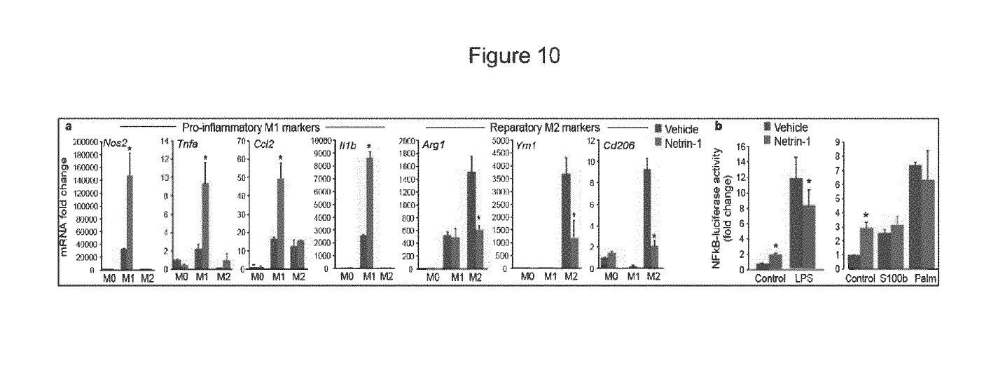

Prior studies demonstrate that Ntn1 diminishes migration of inflammatory cells by acting as a chemorepulsant. This chemorepulsant activity prevents efflux of macrophages from atherosclerotic plaque and adipose tissue (van Gils et al., Arteriosclerosis, Thrombosis, and Vascular Biology 2013; 33(5):911-9; Ramkhelawon et al., Nature Medicine 2014; 20(4):377-84). In preliminary studies it was shown that Ntn1 promotes M1 polarization of macrophages (FIG. 10), nonetheless the effect of Ntn1 on other functions of macrophages has not been explored.

### Ntn1 Inhibits T Cell Adhesion and Promotes Th1 Responses.

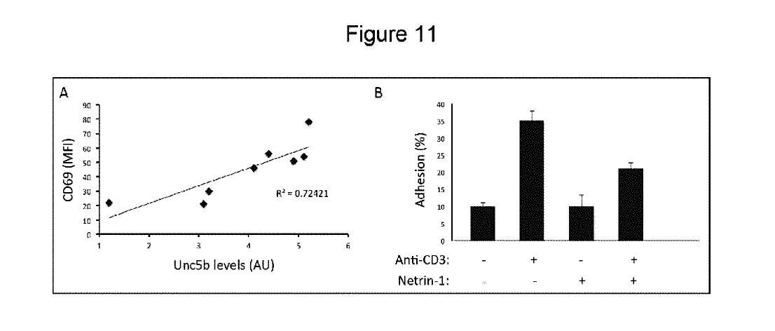

T cells play a critical role in the pathogenesis of inflammatory arthritis. The recruitment of these cells to inflamed joints is regulated by multiple chemokines and chemoattractants. However, the process by which these cells migrate to and from inflamed joints is not completely understood. In order to elucidate the role of Ntn1 in T cell functions, the expression levels of the receptors unc5b and DCC were examined in multiple T cell subsets. Importantly, in human T cells, unc5b, but not DCC, was detected and unc5b levels correlated with other T cell activation markers (FIG. 11A). When these cells were treated with recombinant Ntn1, both static adhesion and adhesion under flow were strongly inhibited (FIG. 11B). Ntn1 also prevented T cell spreading on adherent surfaces, suggesting that Ntn1 effects on T cell adhesion might be mediated by cytoskeletal assembly. The biological significance of these findings and whether Ntn1-inhibited adhesion is relevant to migration to, or from, inflamed joints are not clear. In another experiment T cell responses in inflamed aortic plaques were characterized. In these lesions, Ntn1 deletion resulted in lower levels of Th1 transcription factor (Tbet), Th17 transcription factor (RorγT), and the related cytokines (e.g. IFN-γand IL-17), but higher levels of Th2 transcription factor (Gata3) and Treg transcription factor (Foxp3) and the cytokines (e.g. IL-4), suggesting, for the first time, that, as with macrophages, Ntn1 is a potent T cell polarizing factor.

Ntn1 is a mediator of osteoclast differentiation.

Bone density in mice that had undergone irradiation and bone marrow transplant with Ntn1^{-/-} or wild type (WT) marrow (C57B/6 background for both) was examined. As shown in FIG. 8A, the bone mineral density, as measured by DEXA was significantly increased in mice transplanted with Ntn1^{-/-} marrow, as compared to WT marrow, 20 weeks after marrow transplant. To confirm the effect of Ntn1^{-/-} marrow transplant on bone mass and better understand the effect of the acquired deficiency on bone structure microCT studies of bone were performed, and it was demonstrated that both cortical bone and trabecular bone were increased in the mice transplanted with Ntn1^{-/-} marrow (FIG. 8). Histopathology of bone revealed increased trabecular thickness and few identifiable osteoclasts. To better understand the mechanism by which Ntn1 deficiency leads to increased bone mineral density and cortical and trabecular bone thickness whether osteoclasts from Ntn1^{-/-} mice develop normally was determined. As shown in FIG. 7, in osteoclast precursors isolated from Ntn1-deficient marrow addition of recombinant Ntn1 to the supernates completely reversed the defect in osteoclast differentiation. Moreover, an antibody to unc5b, but not an antibody to DCC, also blocked osteoclast differentiation by precursors from WT mice. In other studies it was shown that little unc5b is expressed on osteoclast precursors before stimulation with RANKL/M-CSF, but that there is marked upregulation of this receptor during osteoclast differentiation. Similarly, Ntn1 expression increased over time in osteoclast precursors. Interestingly, the effect of anti-Ntn1 antibodies on osteoclast differentiation was most marked during the early phases of osteoclast differentiation (FIG. 7). No change in osteoblast differentiation was observed (not shown). These results provide strong evidence that Ntn1 is an autocrine promoter of osteoclast differentiation both in vivo and in vitro.

### Materials and Methods

1. Macrophages.

Intravenous injection of Ntn1 is anti-inflammatory in a number of models of inflammation although this could be explained by binding of Ntn1 to vascular endothelium, which hinders inflammatory cell recruitment (Chen et al., PloS one 2012; 7(9):e46201; Grenz et al., PloS one 2011; 6(5):e14812; Han et al., Investigative Ophthalmology & Visual Science 2012; 53(3):1285-95; Ly et al., Proceedings of the National Academy of Sciences of the United States of America 2005; 102(41):14729-34; Mirakaj et al, Journal of Immunology 2011; 186(1):549-55; Mirakaj et al., American Journal of Respiratory and Critical Care Medicine 2010; 181(8):815-24; Tadagavadi et al., Journal of Immunology 2010;185(6):3750-8; Wang et al., American Journal of Physiology Renal Physiology 2008; 294(4):F739-47; Rosenberger et al., Nature Immunology 2009; 10(2):195-202; Carney et al., Nature Reviews Nephrology 2012. doi: 10.1038/nrneph.2012.228. PubMed PMID: 23090448; Grenz et al., Current Opinion in Critical Care 2012; 18(2):178-85; Mohamed et al., The American Journal of Pathology 2012; Moon et al., Journal of Neuroimmunology 2006; 172(1-2):66-72; Mutz et al., Critical Care 2010; 14(5):R189; Paradisi et al., Proceedings of the National Academy of Sciences of the United States of America 2009; 106(40):17146-51; Rajasundari et al., Laboratory Investigation; A Journal of Technical Methods and Pathology 2011; 91(12):1717-26). Anti-Ntn1 and anti-unc5b, but not anti-DCC, blocking antibodies inhibit macrophage accumulation at sites of wear particle-induced inflammation. It is desirable to expand the understanding of the role of Ntn1 in regulating macrophage migration and chemotaxis, phagocytosis, proliferation and inflammatory responses (e.g. production of cytokines (TNFα, IL-10, IL-6)). In these experiments thioglycollate-elicited murine macrophages, human peripheral blood monocytes and THP-1 human monocytic cell line chemotaxis to CCL19 and CCL21 will be tested in the presence and absence of Ntn1 and in the presence of anti-Ntn1, anti-unc5b and anti-DCC antibodies (Williams et al., Inflammation 2012; 35(2):614-22) using the xCelligence Real Time Cell Migration System. In addition, the interaction of macrophages with synovial fibroblast coated surfaces (Asterand) will be studied to model migration of these cells in the articular compartment. In this assay the cells will be simulated by applying chemokines (e.g. CCL19 and CCL21) with or with pretreatment with Ntn1 and unc5b blocking antibodies.

Previous studies of the effects of Ntn1 on inflammation showed that Ntn1 suppressed IFN-γ-induced M1 macrophages markers and cytokine production, and increased expression of the M2 macrophages marker Cd206/mannose receptor (Ranganathan et al., American Journal of Physiology Renal Physiology 2013; 304(7):F948-57). In addition, Ntn1 inhibited hyperglycemia-induced NFkB activation in tubular epithelial cells (Mohamed et al., The American Journal of Pathology 2012; 181(6):1991-2002). Other studies in BMDM (bone marrow derived macrophages) and THP-1 monocytes have yielded different results. Addition of Ntn1 to classically activated (LPS and IFN-γ) BMDM increased expression of M1 markers, while it blunted expression of M2 markers in alternatively activated (IL-4) BMDM (FIG. 10a). Furthermore, using THP1-Blue NFkB reporter cells (Invitrogen), Ntn1 increased NFkB activation by 3-fold (FIG. 10b). Interestingly, Ntn1 reduced NFkB activation induced by LPS, but had no effect on NFkB-induced by S100b or palmitate (FIG. 10b). These studies strongly suggest a pro-inflammatory function for Ntn1 in macrophages.

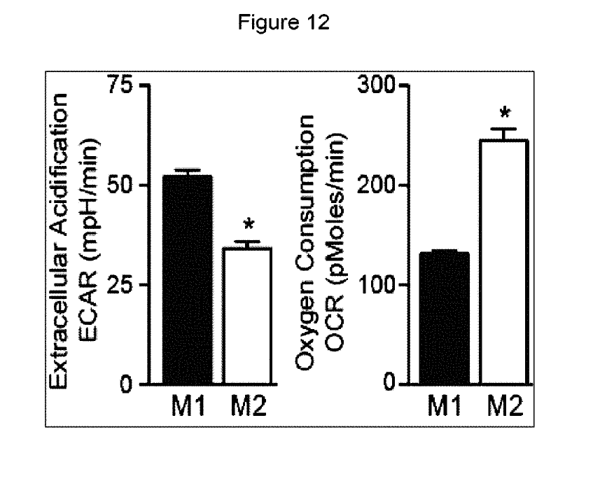

To further investigate the role of Ntn1 in macrophage inflammatory responses, the effect of antibodies to Ntn1 and its receptors on IL-1-stimulated thioglycollate-elicited murine macrophages, human peripheral blood monocytes and THP-1 human monocytoid cell line, and measure TNF-α, IL-10 and IL-6 expression (ELISA) will be determined. The effect of lentiviral-mediated knockdown of Ntn1, unc5b and DCC (vs. stable transduction of lentiviral non targeting RNAs) on these functions in THP-1 cells will be tested as previously described (Williams et al., Inflammation 2012; 35(2):614-22; Bingham et al., Journal of Leukocyte Biology 2010; 87(4):683-90). Transcription factor activation profiling (Signosis) and inflammation antibody arrays (Quantikine) will be performed to identify key transcription factors and inflammatory mediators activated by Ntn1, and based on results, key pathways will be further investigated. Data in FIG. 7 suggest that Ntn1 may enhance M1 inflammatory macrophage polarization and inhibit M2 anti-inflammatory gene expression. Classically activated (LPS+IFN-γ) M1 macrophages have high rates of glucose uptake and lactic acid production, whereas alternatively activated (IL-4) M2 cells rely on fatty acid (3-oxidation and mitochondrial respiration. These parameters can be measured by assessing the extracellular acidification rate (ECAR) and cellular oxygen consumption rates (OCR) of macrophages using the Seahorse XF analyzer (FIG. 12). To test the effect of Ntn1 on macrophage M1/M2 metabolic parameters, mouse peritoneal and human monocyte derived macrophages will be treated with Ntn1 in the presence/absence of relevant receptor blocking antibodies and measure extracellular acidification rate (ECAR) and cellular oxygen consumption rates (OCR) by Seahorse XF analysis.

In addition, how Ntn1 affects the phagocytic capacity of macrophages will be measured by measuring phagocytosis of Ig-coated particles (as previously described Salmon et al., Journal of Immunology 1990;145:2235-40), in the presence and absence of recombinant Ntn1 and antibodies to its receptors (e.g. Unc5b and DCC). To assess the effect of Ntn1 on macrophage proliferation, cells will be treated with Ntn1 in the presence/absence of antibodies to relevant receptors and proliferation will be measured by CyQuant cell proliferation assay. Together, these measures will comprehensively assess the effects of Ntn1 and its receptors on macrophage dynamics and function.

2. T Cells

Ntn1 inhibited T cell adhesion (FIG. 11), and Ntn1 was required for Th1 and Treg polarization. In order to further elucidate and expand these findings the effects of Ntn1 and its receptors on adhesion and migration of different human T cell subsets, under different conditions. PBMC (peripheral blood mononuclear cells) will be separated from whole blood (100-400 ml per unit) from healthy donors (Purchased from New York Blood Center) using Ficoll-Paque density centrifugation. Naïve T cells (CD4⁺CD45RO⁻CD45RA⁺), Th1 cells (CD4⁺CXCR3⁺), Th17 (CD4⁺CXCR3⁻CCR6⁺), and Treg (CD4⁺CD25^{high}) cells will be isolated using the relevant isolation kits (StemCell). Th2 cells (CD4⁺CXCR4⁺) will be isolated using Human CD4⁺ enrichment kit followed by labeling with CXCR4 antibodies (R&D) and selection with EasySep Selection kit (Mediero et al., Science Translational Medicine 2012; 4(135):135ra65). Cytokine profile of each subset will be confirmed by intracellular staining (IFN-γ for Th1; IL-13 for Th2; IL-17 for Th17; and Foxp3 for Treg). Each subset will be labeled with the cell permeable dye CFSE (carboxyfluorescein succinimidyl ester) prior to stimulation with anti-CD3±anti-Ntn1±anti-unc5b antibodies and plating on ICAM-1-coated wells. The number of adherent cells will be quantified by plate-reader. In another set of experiments, adhesion of T cells under flow condition will be studied, an assay designed to mimic interaction of T cells with endothelial cells, a critical step in trafficking of T cells to site of inflammation (Tapia et al., Bulletin of the Hospital for Joint Disease 2014;72(2):148-53). In a complementary experiment the interactions of different T cell subsets with synovial fibroblast-coated surfaces (Asterand) will be studied, to model more accurately the interactions of T cells with the cells of the articular compartment. In this assay the cells will be simulated by applying chemokines (e.g. CCL5 and CXCL12) with or without pretreatment with anti-Ntn1 and anti-un5b blocking antibodies. To directly study LFA-1 affinity regulation on the cell surface in the setting of specific activation states, monoclonal antibodies that bind selectively to the three different conformational structures of integrin LFA-1 will be used.

3. Osteoclasts

Ntn1 is an autocrine mediator of osteoclast formation. The number of osteoclasts (multinucleated TRAP⁺ cells) that develop from Ntn1^{-/-} and WT murine bone marrow in response to RANKL and M-CSF stimulation will be compared, using techniques previously reported (Kara et al., FASEB Journal: Official Publication of the Federation of American Societies for Experimental Biology 2010; 24(7):2325-33). The mRNA expression levels for markers of osteoclast differentiation (TRAP, cathepsin K, NFATc1) will be analyzed by qPCR as previously described (Kara et al., FASEB Journal: Official Publication of the Federation of American Societies for Experimental Biology 2010; 24(7):2325-33). To determine the state of osteoclast differentiation at which Ntn1/unc5b interactions are required, Ntn1^{-/-} bone marrow precursors will be added to recombinant mouse Ntn1 at various time intervals (Day 0, 1, 2, 3, 4) followed by enumeration of osteoclasts and evaluation of actin cytoskeletal rearrangements. Similarly, the effect of Ntn1 deletion on osteoblast differentiation (Alkaline phosphatase, alizarin red stain) and mRNA for osterix and osteocalcin in vitro after incubation in osteogenic medium will be compared. In some experiments recombinant Ntn1, antibodies to Ntn1, DCC or unc5b will be added to cell culture supernates. Although there has been no examination of osteoblasts for either the presence or function of Ntn1, unc5b or DCC on osteoblasts, a preliminary microarray study suggests that the mRNA for these proteins does not change significantly during osteoblast differentiation (not shown), although the baseline expression of these proteins is not yet known. The effect of Ntn1/unc5b/DCC antibodies and Ntn1 or Unc5b deletion will also be tested on osteoblast function, as described above. Identical experiments (using antibodies to Ntn1, unc5b, and DCC) will be performed on cells derived from normal human bone marrow (Lonza) to demonstrate the potential translational relevance of these findings.

Signaling at unc5b for neuronal chemorepulsion is not fully elucidated although recent studies suggest that unc5b signals for chemorepulsion via association with neogenin, a membrane protein (Hata et al., The Journal of Cell Biology 2009; 184(5):737-50). Whether neogenin is expressed by osteoclasts and their precursors (Western Blot) and whether it is associated with unc5b in these cells (immunoprecipitation) will be determined. The role of neogenin in signaling events downstream of Ntn1 /unc5b interactions will be determined.

Mice transplanted with Ntn1^{-/-} marrow have increased bone density (DEXA scan) and increased trabecular and cortical bone thickness (micro CT) after 20 weeks (FIG. 8). To determine whether the change in bone density is due to diminished bone turnover resulting from diminished osteoclast function, increased bone formation by osteoblasts (whether as a result of Ntn1 deficiency or indirectly as a result of diminished suppression of osteoblast function by osteoclasts), or a combination of these effects, bone formation in mice with Ntn1^{-/-} marrow will be examined by use of tetracycline labeling of new bone by IP injection of tetracycline, as previously described (Kara et al., Arthritis and Rheumatism 2010; 62(2):534-41). Bone formation will be examined in these same animals in vivo directly by quantitating the fluorescence of Xenolight Rediject Bone Probe 680, a fluorescent dye that binds to hydroxyapatite in forming bone, by use of the IVIS imaging apparatus, a technique as described (Mediero et al., Arthritis Rheum-US 2012; 64(10):S1062; Mediero et al., Arthritis Rheum-US 2012; 64(10):56-S). The bone phenotype will be determined by histology, DEXA scanning and microCT, of selective Ntn1^{-/-} mice using floxed Ntn1/2.3-cre (osteoblast) and /cathepsin K-cre (osteoclast) mice to determine whether selective loss of Ntn1 in either of these cell types affects bone phenotype. These cre- mice are available commercially. Marrow from these selective knockouts will be studied for osteoclast and osteoblast differentiation and function, as described above, to confirm efficacy of the deletion.

### Results

Ntn1 has previously been shown to inhibit macrophage migration (Ly et al., Proceedings of the National Academy of Sciences of the United States of America 2005; 102(41):14729-34), and this will be confirmed. Nonetheless, it is unknown whether Ntn1 regulates either phagocytosis or cytokine production and it is possible that Ntn1 inhibits (anti-inflammatory), increases (pro-inflammatory) or has no effect on some or any of the functions measured. The net effect of all of Ntn1's effects will determine whether blockade/deletion will be pro- or anti-inflammatory. It is not clear whether the expression levels of unc5b will be similar in all T cell subsets, but there will be correlation between expression levels of this receptor and the ability of Ntn1 to inhibit T cell adhesion. These findings will be more prominent in the adhesion under flow assay and after stimulation with chemokines rather than crosslinking the antigen receptor. Ntn1 will inhibit effector subsets more than naïve and regulatory T cell subsets, further supporting the model in which Ntn1 prevents lymphocyte emigration from an inflamed site, rather then recruitment to the same compartment. Treatment with anti-Ntn1 antibodies will accelerate their interaction with synovial fibroblasts, suggesting that Ntn1 pro-inflammatory effects on synovial inflammation are mediated by blocking emigration from inflamed sites.

Diminished osteoclast formation will correlate with increased bone density/diminished bone turnover in the Ntn1^{-/-} mice. Similarly, the osteoclast-selective Ntn1^{-/-} mice (Ntn1^{fl/fl}/cathepsin-K cre) will have a similar phenotype. Although Ntn1 expression has been reported in osteoblasts (Togari et al., Brain Research 2000; 878(1-2):204-9), the functional role of Ntn1 in osteoblasts is unknown. Nonetheless, based on the bone phenotype of the Ntn1^{-/-} mice it is unlikely that Ntn1^{-/-} osteoblasts will make less osteoid/bone. If the osteoblasts make increased calcified bone (alizarin red staining, in vivo fluorescent uptake of Xenolight Rediject Bone Probe 680) further studies will be performed to determine which receptors (unc5b or DCC) are expressed on osteoblasts and responsible for changes in osteoblast function by use of recombinant Ntn1, antibodies to Ntn1, unc5b and DCC in cultures of differentiating osteoblasts, as well as by use of selective Ntn1^{-/-} mice (Ntn1^{fl/fl}/col 2.3-cre mice).

### Example 4

### Ntn1 Mediates its Cellular Effects by Engaging Receptors that Activate Cytoskeletal Elements

### Background

The three major elements of the cytoskeleton are microfilaments, composed of the protein actin, intermediate filaments (e.g. lamin, keratin, and vimentins), and microtubules, composed of the protein tubulin. The dynamic nature and complexity of these cytoskeletal components emerge from their interactions with additional proteins and regulators such as molecular motors and cross linkers. While the main function of the intermediate filaments is to maintain the shape of the cell, and to anchor organelles, the microfilaments and the microtubule are specifically involved in cellular dynamics such as migration and endocytosis. Microfilaments are the thinnest filaments of the cytoskeleton. They are composed of linear polymers of G-actin subunits, and generate force when the growing end of the filament pushes against a barrier, such as the cell membrane. Actin structures are controlled by the Rho family of small GTPases (e.g. Rho, Rac, and Cdc42). Microtubules are polymers of alpha and beta tubulin. They have a very dynamic behavior, binding GTP for polymerization, and forming the microtubule organization center (MTOC). They are also commonly organized by the centrosome and are associated with centrioles and the protein dynein. Their main functions are intracellular and mitotic spindle regulation.

The most prominent effects of Ntn1 on T cells and macrophages are on their migration and adhesion properties. Actin dynamics are one of the key elements required for normal adhesion, and therefore should be regulated by signaling downstream of Ntn1 and its receptors. Actin dynamics have been proposed to participate extensively from the very first step of leucocyte adhesion, migration and activation. Diverse sets of molecules including receptor-associated kinases, small GTPases and adaptor proteins orchestrate the process of actin polymerization and rearrangement in these cells. The role and regulation of actin in T cell and macrophage dynamics has been the focus of extensive investigation for over a decade. However, how Ntn1 signaling regulates actin dynamics is uncertain. The components of the signaling pathways downstream of Ntn1 leading to altered T cell and macrophage trafficking will be determined.

### Materials and Methods

Many of the studies investigating actin polymerization in T cells and macrophages have been carried out using the fluorescently labeled fungal toxin phalloidin. Using this reagent, it was shown that that Ntn1 inhibited actin polymerization in isolated macrophages. In order to recapitulate these findings in T cells, primary human T cells will be activated by either crosslinking the antigen receptor or by treating the cells with soluble SDF-1, with or without pre-treatment with recombinant Ntn1 or anti-unc5b antibodies, and the morphology of the cells and the distribution of actin polymers using confocal microscopy will be analyzed. Next, the recently F-actin live-cell reporter LifeAct (that binds to F-actin selectively and with sufficiently low affinity that it appears not to affect F-actin while allowing visualization with excellent contrast (Lichius et al., Fungal Biology 2011; 115(6):518-40)) will be used. The cells will be labeled with this reagent and the quantity and localization of F-actin will be recorded in live cells, before and after treatment with Ntn1 and or anti-unc5b antibodies. Similarly, CD11b primary human macrophages will be isolated, stimulated with LPS, and treated with Ntn1 or anti-unc5b antibodies, and actin dynamics in live cells will be recored. These experiments will provide support for the hypothesis that Ntn1 regulates actin dynamics in T cells and macrophages.

To further understand the molecular regulation of actin polymerization downstream of Ntn1, genetic tools will be used. In these studies, cells in which genes coding for proteins involved in actin regulation will be used. Jurkat T cells or THP-1 monocytoid cells will be injected with lentiviruses carrying the relevant shRNA, select stable cells with puromycin, and the knockdown will be validated with qPCR and western blotting. Specifically, the small GTPases RhoA, Rac1, and Cdc42, the exchange factor Vav1, the effector proteins WASp, PKC theta, and Pak1, the scaffold SLP76, and the adaptor and nucleation promoting factor Nck (Lentiviral constructs for murine and human proteins are available from Sigma) will be targeted. The ability of Ntn1 to inhibit T cell or macrophage adhesion and migration will be studied in the absence of these proteins (or after stable transduction with a lentiviral construct expressing a scrambled construct). Cells treated with latrunculin and cytochalasin D will serve as positive controls. The reporter LifeAct will be introduced into these cells, and the cells will be imaged before and during stimulation with recombinant Ntn1. These studies will uncover the differential effect of Ntn1 on either actin nucleation or actin polymerization as well as the contribution of these proteins to signaling downstream of unc5b.

To study if Ntn1 signaling affects other cytoskeletal components such as tubulin dynamics, the following experiments will be performed. The translocation of the microtubule-organizing center (MTOC) is the most characteristic early event that involves the tubulin cytoskeleton in activated T cells and macrophages. It has been shown that the kinase Fyn is the key regulator of tubulin cytoskeleton reorganization (Macurek et al., The Biochemical Journal 2008; 416(3):421-30). First, T cells will be conjugated with antigen presenting cells (RAJI B cells) loaded with SEE, fixed and stained with anti-tubulin antibodies. MTOC translocation will be quantified before and after treatment with anti-unc5b antibodies. Treatment with colchicine will serve as a positive control. Whether signaling downstream of unc5b specifically inhibits Fyn phosphorylation measured by protein phosphoblotting will be determined. Fyn activity will be measured using the in vitro kinase activity assay (R&D). The results of these experiments will support the hypothesis that the effects of Ntn1 on T cell motility are not limited to actin polymerization but also mediated via tubulin dynamics and modulation of Fyn activity. Next tubulin dynamics will be studied in a phagocytosis assay. In this experiment, RAW264.7 cells will be loaded with Zymosan A bio-particles at a ratio of 100 particles/cell. The cells will be incubated for 1 hour at 37° C. to allow particle uptake. Phagocytosis will be stopped by adding cold PBS, followed by fixation and staining as described earlier. The cells will be analyzed using a fluorescence microscope to determine whether the cells have phagocytized the particles, and the effects of blocking antibodies will be quantified. Results can be calculated by determining the phagocytic index (the average number of particles per 100 macrophages).

### Results

Ntn1 will inhibit adhesion and migration by inhibiting both actin polymerization and tubulin reorganization. This will be mediated by inhibiting the small GTPases RhoA and Racl, as well as by recruitment and activation of the kinase Fyn at the adhesion complex. In both B and T lymphocytes, the actin cytoskeleton is envisioned to have a role in steady state segregation of immune receptors and adaptor proteins. Interestingly, F-actin inhibits B cell antigen receptor signals and disruption of F-actin results in B cell activation. In order to understand the role of actin polymerization in unc5b signaling in T cells, T cells will be treated with Latrunculin A (that binds G-actin monomers and prevents polymerization) or with the recently discovered formin inhibitors (that target actin nucleation factors selectively (Lash et al., Cancer Research 2013; 73(22):6793-803), and the expression and distribution of this receptor will be studied before and after stimulation with recombinant Ntn1.

### EXAMPLE 5

### Ntn1 Regulates Cytoskeletal Dynamics and Differentiation in Osteoclasts

### Background

Osteoclast differentiation from committed precursors requires a number of concerted signaling events. Activation of NFkB by RANKL/RANK interactions leads to a complex pattern of new protein expression. Another critical step involves cytoskeletal rearrangements associated with adhesion, migration, podosome formation and fusion (recently reviewed in Itzstein et al., Small GTPases 2011; 2(3):117-30). In many different cell types signaling for cytoskeletal rearrangements and other critical effector mechanisms proceeds via a family of GTPases and in osteoclasts RhoA is one of these critical intermediates (reviewed in Marie et al., Current Osteoporosis Reports 2012. Epub 2012/06/20). Indeed, inhibition of prenylation of RhoA is thought to be the basis for bisphosphonate-mediated osteoclast inhibition (Roelofs et al., Current Pharmaceutical Design 2010; 16(27):2950-60). Because of the known effects of Ntn1 on the cytoskeleton in neuronal cells it is likely that Ntn1 -stimulated signaling at unc5b will be mediated by GTPase-triggered changes in cytoskeletal assembly.

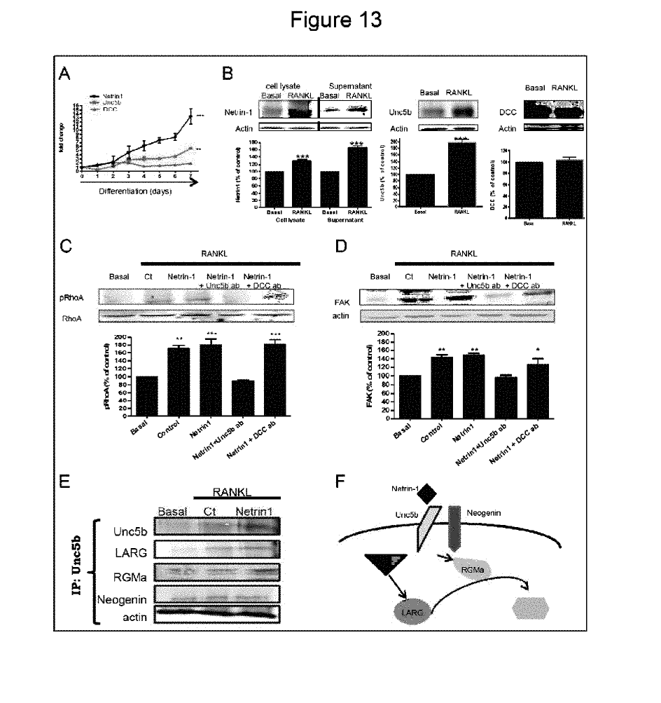

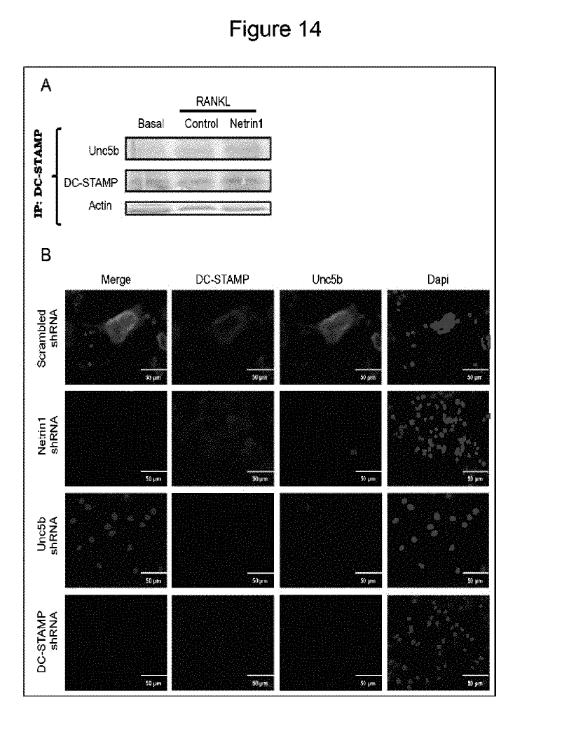

Signaling at unc5b for neuronal chemorepulsion has not been thoroughly investigated although recently it has been suggested that unc5b signals for chemorepulsion via association with neogenin, a membrane protein, that leads to phosphorylation of LARG which, in combination with activation of FAK, leads to RhoA activation and cytoskeletal mobilization (Hata et al., The Journal of Cell Biology 2009; 184(5):737-50). In preliminary studies in RAW264.7 cells that have been induced to undergo osteoclast differentiation by RANKL stimulation, it was observed that Ntn1 activates RhoA and FAK but not Cdc42, a kinase involved in cytoskeletal assembly by different pathways. This activation is abrogated by treating the cells with anti-unc5b antibodies. Moreover, knockdown of Ntn1 expression (using lentiviral-delivery of Ntn1 shRNA) also abrogates RhoA activation and changes LARG distribution in the cell (FIG. 13). These results are consistent with the hypothesis that Ntn1 stimulates osteoclast differentiation by a mechanism similar to that by which Ntn1 acts as a chemorepulsant in neurons. Because these cytoskeletal alterations are most closely associated with cellular fusion during osteoclast differentiation, the association of DC-STAMP with unc5b was examined, and in RAW264.7 cells it was found that DC-STAMP coprecipitates with unc5b and lentiviral-mediated knockdown of unc5b leads to diminished DC-STAMP surface expression and vice versa (FIG. 14). These findings are consistent with the hypothesis that Ntn1-unc5b-mediated events in osteoclast differentiation can be ascribed to their critical role in fusion of precursors into multinucleated osteoclasts. These findings will be confirmed by use of selective knockdown of the signaling molecules involved.

### Materials and Methods

Ntn1 is required for osteoclastogenesis in primary murine cells, primary human cells and RAW264.7 cells. Moreover, Ntn1 -induced changes in FAK and RhoA phosphorylation and activation are similar in primary cells from mice (FIG. 13) and humans as well as in RAW264.7 cells (data not shown). Thus RAW264.7 cells may be used to study signal transduction for osteoclastogenesis for unc5b. Permanent knockdown RAW264.7 cells will be generated for DC-STAMP, unc5b, Ntn1, FAK, RGMA, RhoA, neogenin, and Cdc42 (control), and the capacity of these cells to undergo RANKL-induced osteoclast differentiation as compared to the same cells infected (lentivirus) with scrambled shRNA (TRAP positive multinucleated cells (≧3 nuclei), dentin resorption, F-actin reorganization) will be compared. It will be necessary to co-precipitate unc5b and neogenin to determine whether activation of unc5b regulates the association of these membrane proteins and determine whether neogenin and RGMA co-precipitate as well (neogenin is thought to be a scaffold for activated RGMA during outside-in signaling). Unc5b and DCC, receptors for Ntn1, are dependence receptors, i.e. when not engaged by ligand they stimulate apoptosis (Guenebeaud et al., Molecular Cell 2010; 40(6):863-76; Bagri, Molecular Cell 2010; 40(6):851-3). This phenomenon suggests the hypothesis that in the absence of Ntn1 the developing osteoclasts may undergo apoptosis. To test this, TUNEL staining on Ntn1^{-/-} and WT osteoclasts will be performed to screen for excess apoptosis as previously described (Ramkhelawon et al., Arteriosclerosis, Thrombosis, and Vascular Biology 2013; 33(6):1180-8). If Ntn1^{-/-} cells do undergo apoptosis, the mechanism will be determined by examining dephosphorylation of DAPk (Western Blot) in these cells and by knocking down, as described above, DAPk and PP2a, the signaling molecules responsible for promoting apoptosis downstream of unc5b.

### Results

None of the knockdowns, with the possible exception of DAPk-, PP2a- and Cdc42-deficient cells, will undergo osteoclast differentiation in response to RANKL. The efficacy of the knockdowns will be determined by Western blot analysis of cellular fractions. The ability to easily and permanently knock down specific proteins in the cell has made dissection of signaling pathways much more definitive and straightforward and eliminated the worry that off-target effects of synthetic inhibitors are responsible for the observed effects. If results are equivocal the findings or mechanisms will be confirmed by use of pharmacologic inhibitors.

1. A method for inhibiting, reducing or slowing inflammation or inflammatory arthritis comprising administering to a subject a therapeutically effective amount of an agent effective to inhibit or reduce the biological activity of an axonal guidance protein or a receptor of the axonal guidance protein, or an analog, derivative or a combination thereof

2. The method according to claim 1 wherein the axonal guidance protein is netrin-1.

3. The method according to claim 1 wherein the agent effective to inhibit or reduce the biological activity of the axonal guidance protein or the receptor of the axonal guidance protein is selected from the group consisting of an antibody, a peptide, a nucleic acid, an interfering RNA and a small molecule.

4. The method according to claim 1 wherein the axonal guidance protein is netrin-1 and the axonal guidance protein receptor is unc5b.

5. The method according to claim 1 further comprising administering one or more other agents effective to inhibit inflammation.

6. The method according to claim 5 wherein the one or more other agents is selected from the group consisting of an anti-inflammatory compound, a bisphosphonate and a growth factor.

7. A method for treating an inflammatory arthritis comprising administering to a subject a therapeutically effective amount of an agent effective to inhibit or reduce the biological activity of an axonal guidance protein or a receptor of the axonal guidance protein, or an analog, derivative or a combination thereof

8. The method according to claim 7 wherein the axonal guidance protein is netrin-1.

9. The method according to claim 7 wherein the agent effective to inhibit or reduce the biological activity of the axonal guidance protein or the receptor of the axonal guidance protein is selected from the group consisting of an antibody, a peptide, a nucleic acid, an interfering RNA and a small molecule.

10. The method according to claim 7 wherein the axonal guidance protein is netrin-1 and the axonal guidance protein receptor is unc5b.

11. The method according to claim 7 further comprising administering one or more other agents effective to inhibit inflammation.

12. The method according to claim 11 wherein the one or more other agents is selected from the group consisting of an anti-inflammatory compound, a bisphosphonate and a growth factor.

13. The method according to claim 7 wherein the inflammatory arthritis is selected from the group consisting of rheumatoid arthritis, psoriatic arthritis, and a spondyloarthropathy.

14. A method for inhibiting, reducing or slowing osteoclast differentiation, function, or biological activity comprising administering to a subject a therapeutically effective amount of an agent effective to inhibit or reduce the biological activity of an axonal guidance protein or a receptor of the axonal guidance protein, or an analog, derivative or a combination thereof

15. The method according to claim 14 wherein the axonal guidance protein is netrin-1.

16. The method according to claim 14 wherein the agent effective to inhibit or reduce the biological activity of the axonal guidance protein or the receptor of the axonal guidance protein is selected from the group consisting of an antibody, a peptide, a nucleic acid, an interfering RNA and a small molecule.

17. The method according to claim 14 wherein the axonal guidance protein is netrin-1 and the axonal guidance protein receptor is unc5b.

18. A method for inhibiting or reducing persistence or accumulation of macrophages at an inflamed site or in an inflamed tissue or for promoting or increasing the efflux of macrophages from an inflamed site or inflamed tissue comprising administering to a subject a therapeutically effective amount of an agent effective to inhibit or reduce the biological activity of an axonal guidance protein or a receptor of the axonal guidance protein, or an analog, derivative or a combination thereof

19. The method according to claim 18 wherein the axonal guidance protein is netrin-1.

20. The method according to claim 18 wherein the agent effective to inhibit or reduce the biological activity of the axonal guidance protein or the receptor of the axonal guidance protein is selected from the group consisting of an antibody, a peptide, a nucleic acid, an interfering RNA and a small molecule.

21. The method according to claim 18 wherein the axonal guidance protein is netrin-1 and the axonal guidance protein receptor is unc5b.

22. A method for increasing, stimulating, or promoting lymphocyte cell adhesion comprising administering to a subject a therapeutically effective amount of an agent effective to inhibit or reduce the biological activity of an axonal guidance protein or a receptor of the axonal guidance protein, or an analog, derivative or a combination thereof

23. The method according to claim 22 wherein the axonal guidance protein is netrin-1.

24. The method according to claim 22 wherein the agent effective to inhibit or reduce the biological activity of the axonal guidance protein or the receptor of the axonal guidance protein is selected from the group consisting of an antibody, a peptide, a nucleic acid, an interfering RNA and a small molecule.

25. The method according to claim 22 wherein the axonal guidance protein is netrin-1 and the axonal guidance protein receptor is unc5b.

26. The method according to claim 22 wherein the lymphocyte is a T cell.

27. A method for increasing, stimulating, or promoting lymphocyte efflux from a site of inflammation or from an inflamed tissue comprising administering to a subject a therapeutically effective amount of an agent effective to inhibit or reduce the biological activity of an axonal guidance protein or a receptor of the axonal guidance protein, or an analog, derivative or a combination thereof

28. The method according to claim 27 wherein the axonal guidance protein is netrin-1.

29. The method according to claim 27 wherein the agent effective to inhibit or reduce the biological activity of the axonal guidance protein or the receptor of the axonal guidance protein is selected from the group consisting of an antibody, a peptide, a nucleic acid, an interfering RNA and a small molecule.

30. The method according to claim 27 wherein the axonal guidance protein is netrin-1 and the axonal guidance protein receptor is unc5b.

31. A method for decreasing, inhibiting, or reducing a lymphocyte cellular response comprising administering to a subject a therapeutically effective amount of an agent effective to inhibit or reduce the biological activity of an axonal guidance protein or a receptor of the axonal guidance protein, or an analog, derivative or a combination thereof

32. The method according to claim 31 wherein the axonal guidance protein is netrin-1.

33. The method according to claim 31 wherein the agent effective to inhibit or reduce the biological activity of the axonal guidance protein or the receptor of the axonal guidance protein is selected from the group consisting of an antibody, a peptide, a nucleic acid, an interfering RNA and a small molecule.

34. The method according to claim 31 wherein the axonal guidance protein is netrin-1 and the axonal guidance protein receptor is unc5b.

35. The method according to claim 31 wherein the lymphocyte is a T cell.

36. A pharmaceutical composition comprising an agent effective to inhibit or reduce the biological activity of an axonal guidance protein or a receptor of the axonal guidance protein, or an analog, derivative or a combination thereof

37. The pharmaceutical composition according to claim 36 further comprising one or more other anti-inflammatory agents.
